(19) United States
(12) Patent Application Publication
(10) Pub. No.: US 2005/0187192 A1
(43) Pub. Date: Aug. 25, 2005

(54) Phospholipids for the treatment of infection by togaviruses, herpes viruses and coronaviruses

(75) Inventors: Ronald A. Fleming, Cary, NC (US); Jan V. Hes, Hurdle Mills, NC (US); Yunsheng Huang, Apex, NC (US); Russ H. Read, Rural Hall, NC (US); Susan L. Morris-Natschke, Apex, NC (US); Khalid S. Ishaq, Chapel Hill, NC (US); Louis S. Kucera, Pfaffown, NC (US); Phillip A. Furman, Durham, NC (US)

Correspondence Address: Madeline I. Johnston, Esq.;KING & SPALDING LLP, 45th Floor, 191 Peachtree Street, N.E., Atlanta, GA, 30303, US

(73) Assignee: Kucera Pharmaceutical Company

(21) Appl. No.: 10/783,927

(22) Filed: Feb. 20, 2004

(51) Int. Cl. A61K 31/685

(52) U.S. Cl. 514/78; 514/114

(57) ABSTRACT

Provided are compounds, methods and pharmaceutical compositions for treating a host, especially a human, infected with a togavirus, herpes virus and/or coronavirus, and in particular SARS-CoV, cytomegalovirus or varicella-zoster virus. The method in one embodiment comprises administering to that host an effective amount of an anti-togavirus, anti-herpes virus and/or anti-coronavirus phospholipid or a pharmaceutically acceptable salt or prodrug thereof. The phospholipid compound is, e.g., a 3-alkylamido-2-alkoxypropylphosphocholine compound or salt thereof. The compound may be administered alone or in combination and/or alternation with one or more other anti-viral agents.

### FIELD OF THE INVENTION

This invention is in the area of methods and pharmaceutical compositions for the treatment of viral infections including the treatment of Severe Acute Respiratory syndrome (SARS-CoV) infections and coronaviruses, varicella zoster virus infections, and/or cytomegalovirus infections. The invention includes administering an effective amount of a 3-alkylamido-2-alkoxypropylphospho-choline compound or salt thereof to a host in need thereof.

### BACKGROUND OF THE INVENTION

Viruses

The Togaviridae family of viruses are tightly enveloped virions that are separated into two genera: the alphaviruses and the rubiviruses (Büchen-Osmond, C. (Ed), (2003). 00.026.0.01. Togaviridae. In: ICTVdB—The Universal Virus Database, version 3. ICTVdB Management, The Earth Institute, Biosphere 2 Center, Columbia University, Oracle, Ariz., USA). Among the viruses classified within this genus are the Sindbis virus, Eastern/Western encephalitis viruses, Semliki Forest virus, and Ross River virus.

Togaviruses are simple enveloped viruses, with a genome that consists of a single strand of RNA of positive polarity encapsidated in a protein shell composed of a single species of protein and enveloped by a lipid bilayer derived from the host plasma membrane.

Coronaviruses are a diverse group of large, enveloped, positively stranded RNA viruses that have been implicated in causing a variety of pathological conditions in both humans and other animals (Rota, et al., Sciencexpress, May 1, 2003, pp. 1-10). The coronavirus is composed of an envelope and helical nucleocapsid with club-shaped surface projections that provide “attachment to cells, hemagglutination, and membrane fusion.” (Büchen-Osmond, C. (Ed), (2003). 00.026.0.01. Coronaviridae. In: ICTVdB—The Universal Virus Database, version 3. ICTVdB Management, The Earth Institute, Biosphere 2 Center, Columbia University, Oracle, Ariz., USA). The complete genome is 25,000 to 33,000 nucleotides long and consists of a “single molecule of linear positive-sense single-stranded RNA (Büchen-Osmond, C. (Ed), (2003). 00.026.0.01. Coronaviridae. In: ICTVdB—The Universal Virus Database, version 3. ICTVdB Management, The Earth Institute, Biosphere 2 Center, Columbia University, Oracle, Ariz., USA).

There are three distinct groups of coronaviruses based on their serologic profile, nucleotide sequence, and natural host; groups 1 and 2 contain mammalian viruses, whereas group 3 contains only avian viruses. Coronaviruses typically have narrow host ranges and are fastidious in cell culture. At 30,000 nucleotides (nt), their genome is the largest found in any of the RNA viruses. A recently identified coronavirus is the Severe Acute Respiratory syndrome (SARS) coronavirus (SARS-CoV). Two labs have sequenced the genome of SARS-CoV (˜30,000 nt) which demonstrate that the genome has similarities to coronaviruses but is sufficiently different to represent a new coronavirus (Marra M A et al., Science 2003; 300:1399-1404; Rota et al., Science 2003; 300:1394-1399). The genomes of the two strains described in these reports (Tor2 from Toronto and Urbani from Vietnam) differ by only 8 nt suggesting that the virus remains stable during human passage (Holmes 2003). Comparing the sequence data with that of known coronaviruses, the data indicate that SARS-CoV is not a mutated form of a known coronavirus or a recombinant between known coronaviruses.

The SARS-CoV genome has five major open reading frames (ORFs) encoding for the nucleocapsid (N) protein, the spike (S) protein, the membrane (M) glycoproteins, the envelope (E) proteins, and the replicase polyprotein. These ORFs occur in the same order and are approximately the same size of that found in other coronaviruses (Holmes 2003). The SARS-CoV genome does not encode a hemagglutinin-esterase gene, typically found in group 2 and some group 3 coronaviruses (Rota P A, et al., Science 2003; 300:1394-1399; Marra M A et al., Science 2003; 300:1399-1404) discovered nine ORFs in SARS-CoV not found in other coronaviruses that may encode proteins unique to SARS-CoV.

The clinical features of SARS have been reported (Rainer et al., Brit. Med. J. 2003; 326:1354-1358). The incubation period for the disease is usually from 2 to 7 days. Infection is usually characterized by fever (>38° C.), which is accompanied by one or more signs of respiratory illness (e.g. cough, shortness of breath, difficulty breathing, or hypoxia). Given the nonspecific features of SARS, SARS is difficult to differentiate from other viral infections in its early stages. A rapid diagnostic test, easy to implement, is needed to quickly identify patients with SARS and prevent them from infecting other individuals.

Death from progressive respiratory failure occurs in about 3% to nearly 10% of cases (Poutanen et al., N. Engl. J. Med. 2003 May 15; 348 (20):1995-2005; Tsang et al., N. Engl. J. Med. 2003; 348:1977-1985; and Centers for Disease Control and Prevention. Morbidity and Mortatlity Weekly Report 2003:52(28):664-665). Therapies used initially to treat infected patients consisted of supportive care measures and steroids. Ribavirin, a broad-spectrum antiviral agent with activity against RNA viruses has been administered to SARS patients but has not been observed to be of benefit (Hsu et al., Emerg Infect Dis. 2003 June; 9(6):713-7.). Cinatl et al have reported that interferon beta is effective in vitro against SARS-CoV and could be useful for the treatment of SARS (Cinatl et al., Lancet 2003; 362:293-294). Despite the promise of interferon beta, additional agents with significant activity against SARS-CoV are needed.

To date there are no effective targeted pharmaceutical agents to treat humans infected with SARS CoV. It is therefore an object of the present invention to provide new methods for the treatment of human patients and other hosts infected with SARS.

Herpesviridae is a family of viruses that includes the subfamily alphaherpesvirinae. This subfamily includes the varicelloviruses, including the Varicella-zoster virus (VZV) (human alpha herpes virus) that causes varicella or chicken pox. Herpes viruses include a central linear double stranded DNA, a surrounding capsid, a tegument around the capsid, and an outer lipid envelope with glycoprotein spikes. The Herpesviridae family further includes the subfamily betaherpesvirinae, which includes the infectious human cytomegalovirus.

Antiviral Agents

Examples of antiviral agents that have been identified as active against (+)-RNA viruses include interferon and ribavirin (Battaglia, A. M. et al., Ann. Pharmacother, 2000, 34, 487-494); Berenguer, M. et al. Antivir. Ther., 1998, 3 (Suppl. 3), 125-136).

Ribavirin (1-β-D-ribofuranosyl-1-1,2,4-triazole-3-carboxamide) is a synthetic, non-interferon-inducing, broad spectrum antiviral nucleoside analog. It is sold under the trade names Virazole™ (The Merck Index, 11th edition, Editor: Budavari, S., Merck & Co., Inc., Rahway, N.J., p 1304, 1989); Rebetol (Schering Plough) and Copegus (Roche). U.S. Pat. No. 3,798,209 and RE29,835 disclose ribavirin. Ribavirin is structurally similar to guanosine, and has in vitro activity against several DNA and RNA viruses (Gary L. Davis. Gastroenterology 118:S104-S114, 2000). U.S. Pat. No. 4,211,771 (to ICN Pharmaceuticals) discloses the use of ribavirin as an antiviral agent.

Ribavirin reduces serum amino transferase levels to normal in 40% of patients, but it does not lower viral serum levels (Gary L. Davis. Gastroenterology 118:S104-S114, 2000). Thus, ribavirin alone is not effective in reducing viral RNA levels. Additionally, ribavirin has significant toxicity and is known to induce anemia.

Interferons (IFNs) are compounds that have been commercially available for the treatment of chronic hepatitis for nearly a decade. IFNs are glycoproteins produced by immune cells in response to viral infection. IFNs inhibit viral replication of many viruses, and are known to suppress serum viral RNA to undetectable levels. Additionally, IFN normalizes serum amino transferase levels. Unfortunately, the effects of IFN are temporary and a sustained response occurs in only 8%-9% of patients chronically infected with, for example, HCV (Gary L. Davis. Gastroenterology 118:S104-S 114, 2000).

The combination of IFN and ribavirin for the treatment of viral infection has been reported to be effective in the treatment of IFN naïve patients (Battaglia, A. M. et al., Ann. Pharmacother. 34:487-494, 2000). However, the side effects of combination therapy can be significant and include hemolysis, flu-like symptoms, anemia, and fatigue (Gary L. Davis. Gastroenterology 118:S104-S114, 2000).

Other examples of antiviral agents that have been identified as active against certain (+)-RNA viruses are:
(1) Substrate-based NS3 protease inhibitors (Attwood et al., Antiviral peptide derivatives, PCT WO 98/22496, 1998; Attwood et al., Antiviral Chemistry and Chemotherapy 1999, 10, 259-273; Attwood et al., Preparation and use of amino acid derivatives as anti-viral agents, German Patent Pub. DE 19914474; Tung et al. Inhibitors of serine proteases, PCT WO 98/17679), including alphaketoamides and hydrazinoureas, and inhibitors that terminate in an electrophile such as a boronic acid or phosphonate (Llinas-Brunet et al, PCT WO 99/07734).
(2) Non-substrate-based inhibitors such as 2,4,6-trihydroxy-3-nitro-benzamide derivatives (Sudo K. et al., Biochemical and Biophysical Research Communications, 1997, 238, 643-647; Sudo K. et al. Antiviral Chemistry and Chemotherapy, 1998, 9, 186), including RD3-4082 and RD3-4078, the former substituted on the amide with a 14 carbon chain and the latter processing a para-phenoxyphenyl group;
(3) Thiazolidine derivatives that show relevant inhibition in a reverse-phase HPLC assay with an NS3/4A fusion protein and NS5A/5B substrate (Sudo K. et al., Antiviral Research, 1996, 32, 9-18), especially compound RD-1-6250, possessing a fused cinnamoyl moiety substituted with a long alkyl chain, RD4 6205 and RD4 6193;
(4) Thiazolidines and benzanilides identified in Kakiuchi N. et al. J FEBS Letters 421, 217-220; Takeshita N. et al. Analytical Biochemistry, 1997, 247, 242-246;
(5) A phenanthrenequinone possessing activity against protease in a SDS-PAGE and autoradiography assay isolated from the fermentation culture broth of Streptomyces sp., Sch 68631 (Chu M. et al., Tetrahedron Letters, 1996, 37, 7229-7232), and Sch 351633, isolated from the fungus Penicillium griscofuluum, which demonstrates activity in a scintillation proximity assay (Chu M. et al., Bioorganic and Medicinal Chemistry Letters 9, 1949-1952);
(6) Selective NS3 inhibitors based on the macromolecule elgin c, isolated from leech (Qasim M. A. et al., Biochemistry, 1997, 36, 1598-1607);
(7) Polymerase inhibitors such as nucleotide analogues, gliotoxin (Ferrari R. et al. Journal of Virology, 1999, 73, 1649-1654), and the natural product cerulenin (Lohmann V. et al., Virology, 1998, 249, 108-118);
(8) Antisense phosphorothioate oligodeoxynucleotides (S-ODN) complementary to sequence stretches in the 5′ non-coding region (NCR) of the virus (Alt M. et al., Hepatology, 1995, 22, 707-717);
(9) Inhibitors of IRES-dependent translation (Ikeda N et al., Agent for the prevention and treatment of hepatitis C, Japanese Patent Pub. JP-08268890; Kai Y. et al. Prevention and treatment of viral diseases, Japanese Patent Pub. JP-10101591).
(10) Nuclease-resistant ribozymes (Maccjak, D. J. et al., Hepatology 1999, 30, abstract 995).
(11) Nucleoside analogs have also been developed for the treatment of viral infections.

Various phospholipids have been disclosed for use in therapy applications including treatment of viral infections. U.S. Pat. No. 5,962,437 and PCT Publication WO 96/06620 (Wake Forest University) disclose phospholipid compounds for treating HIV-1, herpes and Hepatitis B viral infections. Phosphoglycerol derivatives for treating viral infections are described in PCT publication WO 91/09602 (Boehringer Mannheim). WO 91/05558 (Boehringer Mannheim) discloses phospholipid antiviral compounds. A phosphocholine moiety has been shown to be an important component for a phospholipid to exhibit antiviral activity (Piantadosi et al., 1991, J. Med. Chem. 34:1408-1414; Krugner-Higby et al., 1995, AIDS Res. & Human Retrovir. 11:705-712).

U.S. Pat. No. 4,444,766 (Boehringer Mannheim) discloses phosphoglycerol derivatives for the treatment of tumors. U.S. Pat. No. 4,562,179 (Fujisawa Pharmaceutical Co., Ltd.) discloses various anti-tumor phospholipid derivatives. U.S. Pat. No. 4,493,832 (Fujisawa Pharmaceutical Co., Ltd.) discloses various phospholipid derivatives. U.S. Pat. No. 4,599,205 (A. Nattermann & Cie) discloses glycerol-phospholipids compounds for the treatment of asthma. U.S. Pat. No. 4,221,732 (A. Nattermann & Cie) discloses phospholipids for, e.g., inhibiting tumor growth.

U.S. Pat. No. 6,429,227 (Alcon Universal Ltd.) discloses various pharmaceutical compositions of hydroxyeicosatetraenoate salts that can be combined with phospholipids for the treatment of dry eye. PCT Publication WO 96/39197 to IMARx Pharmaceutical Corp. discloses phospholipids that are useful as contrast agents for ultrasound. WO 92/22289, assigned to the Regents of the University of California, discloses various phospholipase A2 inhibitors. U.S. Pat. No. 5,116,992 of Societe de Conseils de Recherches discloses glycerol derivatives for the treatment of tumors. WO 90/11079, assigned to Alcon Laboratories, Inc., discloses monoacyl phosphoglycerides for enhancing corneal penetration of ophthalmic drugs. WO 93/08807 discloses glycerophospholipids for antibacterial, antifungal, spermicidal, and virucidal uses.

In view of the severity of diseases associated with togaviruses, coronaviruses and herpes viruses, and their pervasiveness in animals and humans, there is a need for compounds and methods for the treatment of a host infected with these viruses. There is a particular need for compounds and methods of use for the treatment of a host, especially a human, infected with the SARS coronavirus.

### SUMMARY OF THE INVENTION

Compounds, methods and compositions are provided for the treatment of a viral infection in a host, such as a human, in particular a host infected with a togavirus, coronavirus, or herpes virus. In particular, compounds, methods and compositions are provided for the treatment of SARS-CoV, Varicella-zoster virus (VZV) and cytomegalovirus. The methods include administering an effective amount of a 3-alkylamido-2-alkoxypropylphosphocholine compound or salt thereof to a host in need thereof.

In one embodiment, the compounds disclosed herein, and in particular, 3-alkylamido-2-alkoxypropylphosphocholine compounds, can be used in pharmaceutical compositions and methods for the treatment of a togavirus, herpes virus and/or coronavirus infection.

In a particular embodiment, the compounds disclosed herein, and in particular, 3-alkylamido-2-alkoxypropylphosphocholine compounds, can be used in compositions and methods for the treatment of a herpes virus, such as varicella zoster virus or cytomegalovirus.

In a particular embodiment, the compounds disclosed herein, and in particular, 3-alkylamido-2-alkoxypropylphosphocholine compounds, can be used in compositions and methods for the treatment of a coronavirus, such as SARS-CoV.

Compounds useful in methods and compositions for the treatment of infections caused by a togavirus, herpes virus and/or coronavirus, including varicella zoster virus, cytomegalovirus, or SARS-CoV, include phospholipid compounds of the formula: 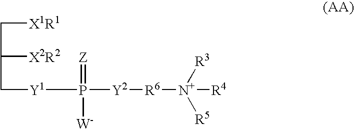
in any of its tautomeric, stereoisomeric or enantiomeric forms;
or a pharmaceutically acceptable salt and/or prodrug thereof, wherein:
R¹ is alkyl, such as C₁-C₂₂ alkyl or C₁-C₁₂ alkyl, or is alkenyl or alkynyl, and is optionally substituted (e.g., from 1 to 5 times) with —OH, —COOH, oxo, or amino;
R² is alkyl, such as C₁-C₂₂ alkyl, or is alkenyl or alkynyl, and is optionally substituted (e.g., from 1 to 5 times) with —OH, —SH, oxo, amine, amide, —COOH, or ester;
X¹ and X² are independently amide, carbonylamino, aminocarbonyl, ureido, ester, amine, hydrazine, —NR′—NR—, —NHC(O)—, —NR′C(O)—, —N(CH₃)C(O)—, —C(O)NH—, —C(O)NR′—, —C(O)N(CH₃)—, —NH—, —NR′—, —N(CH₃)—, —(C═NH)—, —(C═NR′)—, —O(C═NH)—, —O(C═NR′)—, —(C═NH)O—, —(C═NR′)O—, —S(C═NH)—, —S(C═NR′)—, —(C═NH)S—, —(C═NR′)S—, —O(C═NH)O—, —S(C═NH)O—, —O(C═NH)S—, —S(C═NH)S—, —O(C═NR′)O—, —S(C═NR′)O—, —O(C═NR′)S—, —S(C═NR′)S—, —C(O)—, —OC(O)—, —C(O)O—, —OC(O)O—, —SC(O)—, —C(O)S—, —SC(O)O—, —OC(O)S—, —SC(O)S—, —NHC(O)NH—, —NHC(O)NR—′, —N(R′)C(O)NH—, —N(R′)C(O)NR″—, —NHC(S)—, —NR′C(S)—, —N(CH₃)C(S)—, —C(S)NH—, —C(S)NR′—, —C(S)N(CH₃)—, —C(S)—, —OC(S)—, —C(S)O—, —OC(S)O—, —SC(S)—, —C(S)S—, —SC(S)O—, —OC(O)S—, —SC(S)S—, —NHC(S)NH—, —NHC(S)NR′—, —NR′C(S)NH—, —NR′C(S)NR″—, —O—, —S—, —S(O)—, —(SO₂), sulphinyl, or sulphonyl;
Y¹ and Y² are selected independently from the group consisting of O, S or Se;
Z is O, S, Se, NH, or NR′;
W is O, S, NH, or NR′;
R⁶ is alkyl, such as C₁-C₆ alkyl, and in particular methyl or ethyl or is alkenyl or alkynyl; and
R³, R⁴ and R⁵ are independently alkyl, such as C₁ to C₆ alkyl, e.g., methyl or ethyl, or R³ and R⁴ together form a heterocyclic ring, for example having three, four, five, six or seven members and R⁵ is an alkyl, such as a C₁ to C₆ alkyl, preferably methyl or ethyl; and
R′ and R″ are independently alkyl, alkenyl, alkynyl, saturated or unsaturated cycloalkyl, aryl, heteroaryl, or heterocyclic.

In a subembodiment, compounds useful in the methods and compositions for the treatment of a togavirus, herpes virus and/or coronavirus infection, and in particular an infection of varicella zoster virus, cytomegalovirus, or SARS-CoV, are compounds of the formula below: 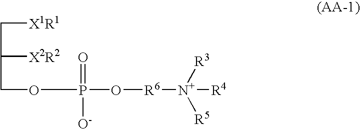
in any of its tautomeric, stereoisomeric or enantiomeric forms;
or a pharmaceutically acceptable salt and/or prodrug thereof, wherein:
R¹ is C₁-C₁₂ alkyl, C₂-C₁₂ alkenyl or C₂-C₁₂ alkynyl optionally substituted from 1 to 5 times with —OH, —COOH, oxo, or amine;
X¹ is —NHC(O)—, —N(CH₃)C(O)—, —C(O)NH—, —C(O)N(CH₃)—, —NH— or —N(CH₃)—;
R² is C₁-C₁₂ alkyl, C₂-C₁₂ alkenyl or C₂-C₁₂ alkynyl optionally substituted from 1 to 5 times with —OH, —COOH, oxo, or amine;
X² is —NHC(O)—, —N(CH₃)C(O)—, —C(O)N(CH₃)—, —S—, —SO—, —SO₂—, —O—, —NH— or —N(CH₃)—;
R⁶ is C₁-C₆ alkyl, C₂-C₆ alkenyl or C₂-C₆ alkynyl; and
R³, R⁴ and R⁵ are independently methyl or ethyl; or
R³ and R⁴ together form a heterocyclic ring having five or six members and R₅ is methyl or ethyl.

In one embodiment, it has been surprisingly found that phospholipids with lower alkyl substituents and optionally a phosphocholine substituent, such as the compounds of formula (AA-1) with lower alkyl chains in the R¹ and/or R² positions, and in particular the R² position, are active against viral infections, such as togavirus, herpes virus and/or coronavirus infection, and in particular an infection of varicella zoster virus, cytomegalovirus, or SARS-CoV.

Therefore, in one subembodiment, compounds useful in the methods and compositions for the treatment of a togavirus, herpes virus and/or coronavirus infection, and in particular an infection of varicella zoster virus, cytomegalovirus, or SARS-CoV, are compounds of the formula below: 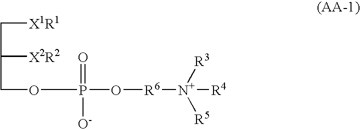
in any of its tautomeric, stereoisomeric or enantiomeric forms;
or a pharmaceutically acceptable salt and/or prodrug thereof, wherein:
X¹ is —NHC(O)—, —N(CH₃)C(O)—, —C(O)NH—, —C(O)N(CH₃)—, —NH— or —N(CH₃)—;
X² is —NHCO—, —N(CH₃)C(O)—, —C(O)N(CH₃)—, —S—, —SO—, —SO₂—, —O—, —NH— or —N(CH₃)—;
R¹ is C₁-C₂₂ alkyl, C₂-C₂₂ alkenyl or C₂-C₂₂ alkynyl;
R² is C₁-C₁₂ alkyl, C₂-C₁₂ alkenyl or C₂-C₁₂ alkynyl;
wherein at least one of R¹ and R² independently is C₁-C₇ alkyl e.g., C₂ or C₃ alkyl, C₂-C₇ alkenyl or C₂-C₇ alkynyl;
R⁶ is C₁-C₆ alkyl, C₂-C₆ alkenyl or C₂-C₆ alkynyl; and
R³, R⁴ and R⁵ are independently methyl or ethyl; or
R³ and R⁴ together form a heterocyclic ring having five or six members and R₅ is methyl or ethyl.

In an alternative embodiment, one or more alkyl groups disclosed herein are substituted.

In a particular subembodiment, the compound useful composition and methods for the treatment of an infection of a togavirus, herpes virus or coronavirus,and in particular, varicella zoster virus, cytomegalovirus, or SARS-CoV, is a compound of the formula AA-1, in any of its tautomeric, stereoisomeric or enantiomeric forms; or a pharmaceutically acceptable salt and/or prodrug thereof, wherein:
X¹ is —NHC(O)—;
X² is —S— or —O—;
R¹ and R² are independently an unbranched, saturated C₁ to C₂₂ alkyl group;
at least one of R¹ and R² is an unbranched, saturated C₁ to C₅ alkyl group;
R⁶ is an unbranched saturated C₂ to C₆ alkyl group; and
R³, R⁴ and R⁵ are independently methyl or ethyl.

In another embodiment, in the compound of formula AA-1:
X¹ is —NHC(O)—;
X² is —O—;
R¹ and R² are independently an unbranched, saturated C₁ to C₂₂ alkyl group;
at least one of R¹ and R² is an unbranched, saturated C₁ to C₅ alkyl group;
R⁶ is an unbranched saturated C₂ to C₆ alkyl group; and
R³, R⁴ and R⁵ are independently methyl or ethyl.

In another embodiment, in the compound of formula AA-1:
X¹ is —NHC(O)—;
X² is —O—;
R¹ is an unbranched, saturated C₁ to C₂₂ alkyl group, e.g., C₇-C₁₁;
R² is an unbranched, saturated C₁ to C₅ alkyl group;
R⁶ is CH₂CH₂; and
R³, R⁴ and R⁵ are independently methyl or ethyl.

In a particular embodiment, a method for treating a host infected with a coronavirus, herpes virus or togavirus is provided, comprising administering an anti-viral effective amount of a compound, or a pharmaceutically acceptable salt or prodrug thereof, having a structure of Formula AA-1: 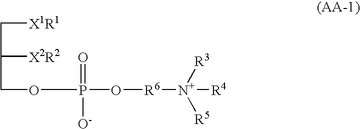
wherein:
X¹ is —NHC(O)—;
X² is —O—;
R¹ is —C₁-C₂₂ alkyl;
R² is —C₁-C₂₂ alkyl;
R⁶ is —CH₂CH₂; and
R³, R⁴ and R⁵ are methyl.

In a particular embodiment of the compound of formula AA-1:
R¹ is —CH₃, —CH₂CH₃, —CH₂CH₂CH₃, —CH₂CH₂CH₂CH₃, —CH₂CH₂CH₂CH₂CH₃, —(CH₂)₅CH₃, —(CH₂)₆CH₃, —(CH₂)₇CH₃, —(CH₂)₈CH₃, —(CH₂)₉CH₃, —(CH₂)₁₀CH₃, —(CH₂)₁₁CH₃, —(CH₂)₁₂CH₃ or —(CH₂)₁₃CH₃; and
R² is —CH₃, —CH₂CH₃, —CH₂CH₂CH₃, —CH₂CH₂CH₂CH₃, —CH₂CH₂CH₂CH₂CH₃, —(CH₂)₅CH₃, —(CH₂)₆CH₃, —(CH₂)₇CH₃, —(CH₂)₈CH₃, —(CH₂)₉CH₃, —(CH₂)₁₀CH₃, —(CH₂)₁₁CH₃, —(CH₂)₁₂CH₃ or —(CH₂)₁₃CH₃.

In one embodiment of the compound of formula AA-1, e.g., when the host is infected with a coronavirus, such as SARS-CoV, and:
R¹ is —(CH₂)₈CH₃, —(CH₂)₉CH₃, —(CH₂)₁₀CH₃, —(CH₂)₁₁ CH₃; —(CH₂)₁₂CH₃, or —(CH₂)₁₃CH₃; and
R² is CH₃, —CH₂CH₃, —CH₂CH₂CH₃, —CH₂CH₂CH₂CH₃, —CH₂CH₂CH₂CH₂CH₃, —(CH₂)₅CH₃, —(CH₂)₆CH₃, or —(CH₂)₇CH₃;
   In another embodiment of the compound of formula AA-1, the host is infected with a herpes virus, such as varicella zoster virus, and:
R¹ is —(CH₂)₅CH₃, —(CH₂)₆CH₃, —(CH₂)₇CH₃, —(CH₂)₈CH₃, —(CH₂)₉CH₃, —(CH₂)₁₀CH₃, —(CH₂)₁₁CH₃, or —(CH₂)₁₂CH₃; and
R² is —(CH₂)₆CH₃, —(CH₂)₇CH₃, —(CH₂)₈CH₃, —(CH₂)₉CH₃, —(CH₂)₁₀CH₃, —(CH₂)₁₁CH₃, —(CH₂)₁₂CH₃, or —(CH₂)₁₃CH₃;

In a specific embodiment, compounds useful in methods and compositions for the treatment of an infection of a togavirus, herpes virus and/or coronavirus infection, and in particular varicella zoster virus, cytomegalovirus, or SARS-CoV, are provided, wherein the compound is: 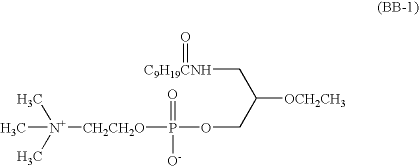
in any of its tautomeric, stereoisomeric or enantiomeric forms; or a pharmaceutically acceptable salt and/or prodrug thereof.

In another specific embodiment, a compound for use in methods and compositions for the treatment of a togavirus, herpes virus and/or coronavirus infection, and in particular a varicella zoster virus, cytomegalovirus, or SARS-CoV infection, is provided having the structure: 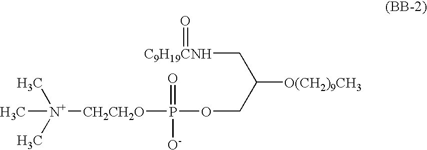
in any of its tautomeric, stereoisomeric or enantiomeric forms; or a pharmaceutically acceptable salt and/or prodrug thereof.

In another specific embodiment, the compound useful in methods and compositions for the treatment of a togavirus, herpes virus and/or coronavirus infection is the compound: 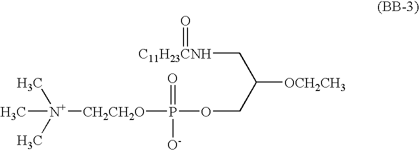
in any of its tautomeric, stereoisomeric or enantiomeric forms; or a pharmaceutically acceptable salt and/or prodrug thereof.

In a particular embodiment, the compound BB-3 is useful in methods and compositions for the treatment of a herpes virus, such as varicella zoster virus or cytomegalovirus, or for the treatment of a coronavirus, such as SARS-CoV.

In another specific embodiment, the compound for the treatment of a togavirus, herpes virus and/or coronavirus infection is the compound: 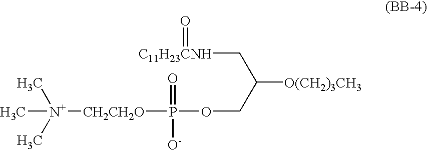
in any of its tautomeric, stereoisomeric or enantiomeric forms; or a pharmaceutically acceptable salt and/or prodrug thereof.

The compound BB-4 can be used for example in methods and compositions for the treatment of a herpes virus, such as varicella zoster virus or cytomegalovirus,or for the treatment of a coronavirus, such as SARS-CoV.

Also useful for the treatment of a togavirus, herpes virus, and/or coronavirus are compounds, or pharmaceutically acceptable salts and prodrugs thereof, of Formula I: 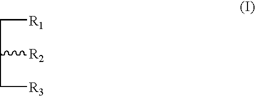
wherein:
R₁ is —NHC(O)Y, where Y is C₁-C₂₂ alkyl, C₂-C₂₂ alkenyl, or C₂-C₂₂ alkynyl;
R₂ is —OX, where X is C₁-C₂₂ alkyl, C₂-C₂₂ alkenyl, or C₂-C₂₂ alkynyl; and
R₃ is phosphocholine (—OPO₃⁻CH₂CH₂N⁺(CH₃)₃).
   Embodiments of compounds of Formula I:
In one embodiment, Y is —C₉H₁₉ or —C₁₁H₂₃.
In another embodiment, X is —C₂H₅ or —C₁₀H₂₁.
   In another embodiment:
   Y is C₁-C₁₄ alkyl, C₂-C₁₄ alkenyl, or C₂-C₁₄ alkynyl; and
   X is C₁-C₁₄ alkyl, C₂-C₁₄ alkenyl, or C₂-C₁₄ alkynyl.
      In another embodiment:
   Y is C₁-C₅ alkyl, C₂-C₅ alkenyl, or C₂-C₅ alkynyl; and
      In another embodiment:
   X is C₁-C₅ alkyl, C₂-C₅ alkenyl, or C₂-C₅ alkynyl.
      In a further embodiment:
   Y is —C₁₁H₂₃, —C₁₀H₂₁, or —C₉H₁₉; and
   X is —CH₂CH₃, —(CH₂)₂CH₃, —(CH₂)₃CH₃, or —CH₁₀CH₂₁.

In one embodiment, Y is —C₁₁ H₂₃ and X is C₁-C₅ alkyl.

In another embodiment, Y is —C₉H₁₉ and X is C₁-C₅ alkyl.

Compounds of Formula I can be used for example in methods and compositions for the treatment of a herpes virus, such as varicella zoster virus or cytomegalovirus, or for the treatment of a coronavirus, such as SARS-CoV.

Also useful for the treatment of a togavirus, herpes virus, and/or coronavirus are compounds, or pharmaceutically acceptable salts and prodrugs thereof, of Formula II: 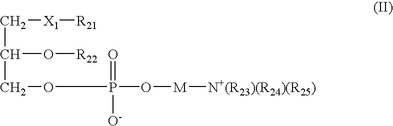
wherein:
M is C₂-C₄ alkyl;
X₁ is —S—, —O—, —NH—, or —NHC(O)—;
R₂₁ is C₁-C₂₀ straight chain alkyl or C₂-C₂₀ straight chain alkylene containing not more than four double bonds, or aryl;
R₂₂ is hydrogen, methyl, or ethyl, or in another embodiment, R₂₂ is C₁-C₂₀ straight chain alkyl or C₂-C₂₀ straight chain alkylene containing not more than four double bonds, or aryl; and

R₂₃, R₂₄, and R₂₅ are each independently hydrogen, methyl, ethyl, propyl, or isopropyl.

For example, R₂₃, R₂₄, and R₂₅ are methyl.

In a subembodiment of Formula II:

M is —CH₂CH₂—;
X₁ is —S—, —O—, —NH—, or —NHC(O)—;
R₂₁ is C₂-C₁₆ straight chain alkyl, or —C₂-C₁₆ straight chain alkylene containing not more than one double bond;
R₂₂ is C₂-C₁₆ straight chain alkyl, or —C₂-C₁₆ straight chain alkylene containing not more than one double bond; and

R₂₃, R₂₄, and R₂₅ are each independently hydrogen or methyl.

In another subembodiment of Formula II:

R₂₂ is C₁-C₅ straight chain alkyl, or —C₂-C₅ straight chain alkylene containing not more than one double bond.
   In one subembodiment, of the compound of Formula II,
R₂, is a C₂-C₁₆ straight chain alkyl or C₂-C₁₆ straight chain alkylene containing not more than one double bond; and
R₂₂ is a C₂-C₅ straight chain alkyl or C₂-C₅ straight chain alkylene containing not more than one double bond.

In another embodiment, R₂₁ is —C₉-C₁₂ alkyl, and R₂₂ is —C₁-C₁₂ alkyl.

In another embodiment, R₂₁ is —C₉-C₁₂ alkyl, and R₂₂ is —C₁-C₅ alkyl.

In another embodiment, R₂, is —C₉-C₁₂ alkyl, and R₂₂ is —C₈-C₁₂ alkyl.

In another embodiment of Formula II:
M is —CH₂CH₂—;
X₁ is —NHC(O)—;
R₂₁ is a C₁₆-C₁₈ straight chain alkyl or —C₂-C₁₈ straight chain alkylene containing not more than one double bond;
R₂₂ is hydrogen, methyl, or ethyl; and

R₂₃, R₂₄, and R₂₅ are each independently hydrogen or methyl.

In another embodiment of Formula II:
M is —CH₂CH₂—;
X₁ is —NHC(O)—;
R₂₁ is —C₁₁H₂₃ or —C₉H₁₉;
R₂₂ is —C₂H₅ or —C₁₀H₂₁; and

R₂₃, R₂₄ and R₂₅ are methyl.

Compounds of Formula II can be used for example in methods and compositions for the treatment of a herpes virus, such as varicella zoster virus or cytomegalovirus, or for the treatment of a coronavirus, such as SARS-CoV.

Further compounds, or pharmaceutically acceptable salts and prodrugs thereof, useful for the treatment of a togavirus, herpes virus, and/or coronavirus are compounds of Formula III: 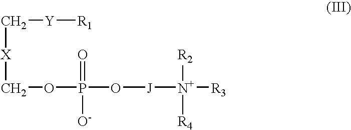
wherein:
Y is —S—, —O—, —NH—, —N(CH₃)—, —NHC(O)—, or —N(CH₃)C(O)—;
R₁ is C₁₄-C₁₈ alkyl, C₁₄-C₁₈ alkenyl, C₁₄-C₁₈ alkynyl, or aryl, or optionally C₁-C₁₈ alkyl, C₂-C₁₈ alkenyl, or C₂-C₁₈ alkynyl;
X is a covalent bond or methylene that is optionally substituted with a hydroxyl, C₁-C₂₀ alkyl, —O—(C₁-C₂₀ alkyl), —S—(C₁-C₂₀ alkyl), —C(O)N(C₁-C₂₀ alkyl), C₂-C₂₀ alkenyl, —O—(C₂-C₂₀ alkenyl), —S—(C₂-C₂₀ alkenyl), —(C(O)N(C₂-C₂₀ alkenyl), C₂-C₂₀ alkynyl, —O—(C₂-C₂₀ alkynyl), —S—(C₂-C₂₀ alkynyl) or —(C(O)N(C₂-C₂₀ alkynyl);
J is a C₁-C₄ alkyl optionally substituted from one to three times with methyl or ethyl; and
R₂, R₃, and R₄ are independently hydrogen or C₁-C₃ alkyl.

In one embodiment of the compound of Formula III:
Y is —NHC(O)—;
R₁ is —C₆-C₁₈ alkyl;
X is —CH—O—(C₁-C₁₈ alkyl or alkenyl);
J is —CH₂CH₂—; and

R₂, R₃, and R⁴ are each methyl.

In another embodiment of the compound of Formula III, X is —CH—O—(C₁-C₅ alkyl) or —CH—O—(C₂-C₅ alkenyl);

In another embodiment of the compound of Formula III, R₁ is —C₈-C₁₂ alkyl and X is —CH—O—(C₁-C₅ alkyl) or —CH—O—(C₂-C₅ alkenyl).

In another embodiment of the compound of Formula III, R₁ is —C₈-C₁₂ alkyl and X is —CH—O—(C₈-C₁₂ alkyl) or —CH—O—(C₈-C₁₂ alkenyl).

In one embodiment, of the compound of Formula III, R₁ is —C₁₁H₂₃ and X is —C(H)(O—C₁-C₅ alkyl)-or —C(H)(O—C₁-C₅ alkenyl)-

In one embodiment, of the compound of Formula III, R₁ is —C₉H₁₉ and X is —C(H)(OC₂H₅)—.

In one embodiment, of the compound of Formula III, R₁ is —C₉H₁₉ and X is —C(H)(OC₁₀H₂₁)—.

Compounds of Formula III can be used for example in methods and compositions for the treatment of a herpes virus, such as varicella zoster virus or cytomegalovirus, or for the treatment of a coronavirus, such as SARS-CoV.

Further useful compounds include compounds, or pharmaceutically acceptable salts and prodrugs thereof, for the treatment of a togavirus, herpes virus, and/or coronavirus of Formula IV: 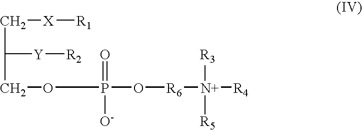
wherein:
R₁ is a C₆-C₁₈ alkyl, C₆-C₁₈ alkenyl, or C₆-C₁₈ alkynyl that is optionally substituted from 1 to 5 times with —OH, —COOH, oxo, amino, or aryl;
X is —NHC(O)—, —N(CH₃)C(O)—, —C(O)NH—, —C(O)N(CH₃)—, —S—, —S(O)—, —(SO₂)—, —O—, —NH—, or —N(CH₃)—;
R₂ is C₁-C₁₄ alkyl, C₂-C₁₄ alkenyl, or C₂-C₁₄ alkynyl that is optionally substituted from 1 to 5 times with —OH, —COOH, oxo, amino, or aryl;
Y is —NHC(O)—, —N(CH₃)C(O), —C(O)NH—, —C(O)N(CH₃)—, —S—, —S(O)—, —(SO₂)—, —O—, —NH—, —N(CH₃)—, or —OC(O)—;
R₆ is a C₂-C₆ alkyl; C₂-C₆ alkenyl, or C₂-C₆ alkynyl; and
R₃, R₄, and R₅ are independently methyl or ethyl, or R₃ and R₄ together form an aliphatic or heterocyclic ring having five or six ring atoms and R₅ is methyl or ethyl.

In one embodiment of the compound of Formula IV:
R₂ is C₁-C₁₄ alkyl, C₂-C₁₄ alkenyl, or C₂-C₁₄ alkynyl;
R₆ is CH₂CH₂; and
R₃, R₄, and R₅ are each CH₃.

In one embodiment of the compound of Formula IV, R₂ is —C₁-C₅ alkyl or —C₁-C₅ alkenyl;

In another embodiment of the compound of Formula IV, R₁ is —C₈-C₁₂ alkyl and R₂ is —C₁-C₁₂ alkyl.

In another embodiment of the compound of Formula IV, R₁ is —C₈-C₁₂ alkyl and R₂ is —C₁-C₅ alkyl.

In another embodiment of the compound of Formula IV, R₁ is —C₈-C₁₂ alkyl and R₂ is —C₈-C₁₂ alkyl.

In another embodiment of the compound of Formula IV:

X is —NHC(O)—, —N(CH₃)C(O)—, —C(O)NH—, or —C(O)N(CH₃)—; and

Y is —O—, —NH—, or —N(CH₃)—.

Compounds of Formula IV can be used for example in methods and compositions for the treatment of a herpes virus, such as varicella zoster virus or cytomegalovirus, or for the treatment of a coronavirus, such as SARS-CoV.

The compounds disclosed herein may be administered, e.g., orally, intravenously, parentally, intradermally, subcutaneously, topically, or by inhalation, optionally with a suitable carrier.

The invention also provides a method for treating a host infected with a togavirus, herpes virus, and/or coronavirus that includes administering to a host in need thereof an effective amount of a compound, or pharmaceutically acceptable salt or prodrug thereof, having a structure disclosed herein.

The invention in particular provides a method of treatment of a herpes virus, such as varicella zoster virus or cytomegalovirus,or for the treatment of a coronavirus,such as SARS-CoV, comprising administering to a host in need thereof an anti-viral effective amount of a compound, or pharmaceutically acceptable salt or prodrug thereof, having a structure disclosed herein.

The invention in particular provides a method for treating a host infected with SARS that includes administering an effective amount of a compound, or pharmaceutically acceptable salts or prodrugs thereof, having a structure disclosed herein.

The invention also provides a pharmaceutical composition for the treatment of a herpes virus, such as varicella zoster virus or cytomegalovirus,or for the treatment of a coronavirus, such as SARS-CoV, comprising an anti-viral effective amount of a compound, or pharmaceutically acceptable salt or prodrug thereof, having the structure disclosed herein.

The invention further provides a pharmaceutical composition for treating a host infected with SARS comprising an effective amount of a compound, or pharmaceutically acceptable salt or prodrug thereof, having a structure disclosed herein.

The invention further provides a method of inhibiting togavirus, herpes virus, and/or coronavirus viral replication in a cell, comprising administering to the cell, in an amount effective to inhibit replication of the infectious virus in the cell, a compound, or pharmaceutically acceptable salt or prodrug thereof, having a structure disclosed herein.

The invention also provides a pharmaceutical composition or kit comprising a compound, or pharmaceutically acceptable salt or prodrug thereof, of a formula disclosed herein.

The active compounds can be administered in combination, alternation or sequential steps with another anti-viral agent. In preferred embodiments, an anti-viral compound exhibits an EC₅₀ of 10-15 μM, or less than 1-5 μM.

It is intended that the active phospholipids compounds of the present invention include phospholipids of the general formulas disclosed herein or a pharmaceutically acceptable salt or prodrug thereof, in any of its tautomeric, stereoisomeric or enantiomeric forms. The invention includes a pharmaceutical composition comprising one or more of these compounds; a medicament comprising one or more of these compounds; and a process for preparing such a composition and/or medicament, as well as methods of treatment using such compounds and compositions.

The invention also provides a method of inhibiting togavirus, herpes virus or coronavirus replication in a cell, such as a mammalian cell, comprising administering to the cell, in an amount effective to inhibit viral replication in the cell, a compound, or a pharmaceutically acceptable salt or prodrug thereof, having a structure of Formula I as defined herein, or any other compound or formula as defined herein. For example, the compound may be: 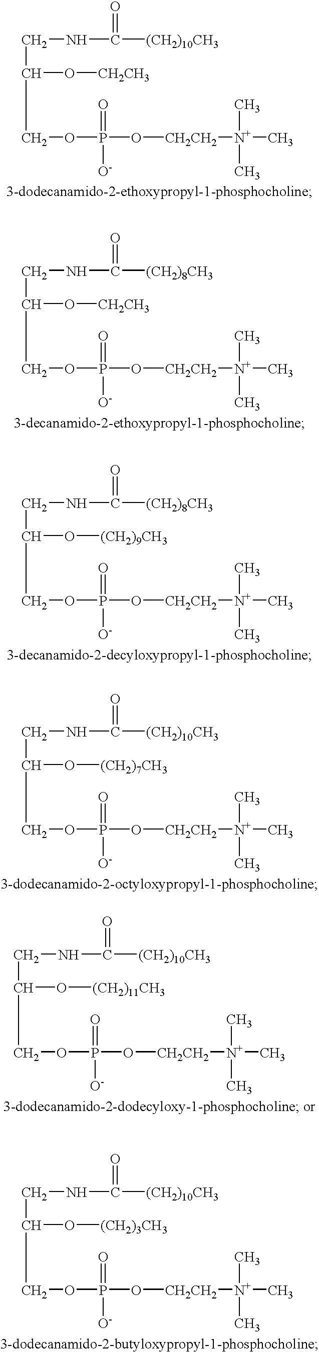
or a combination thereof.

### BRIEF DESCRIPTION OF THE DRAWINGS

FIG. 1 shows a phylogenetic tree of the SARS-associated coronavirus.

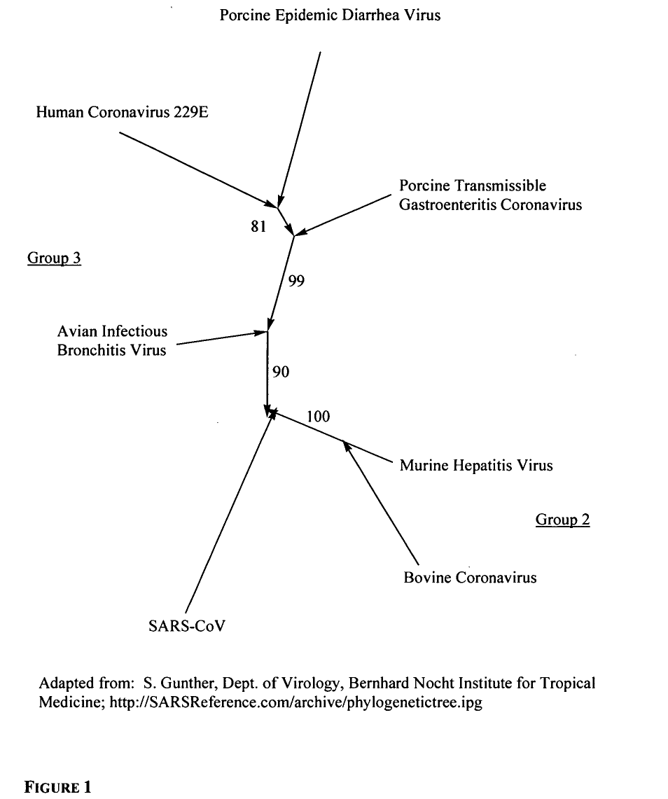

FIG. 2 illustrates a process that may be used generally for obtaining a 3-alkylamido-2-alkoxypropylphosphocholine.

### DETAILED DESCRIPTION OF THE INVENTION

The present invention provides compounds, methods and compositions for the treatment of a host, and in particular a human or an animal, infected with an enveloped (+)- stranded RNA virus, such as a togavirus or coronavirus. The invention further provides methods, compounds and compositions for treatment of a herpes viral infection in a host. This treatment includes administering an effective amount of an anti-viral phospholipid as described herein, or a pharmaceutically acceptable salt or prodrug thereof, optionally in a pharmaceutically acceptable carrier. The compounds used in this invention may possess antiviral activity, or may be metabolized to a compound that exhibits such activity.

Methods and compositions for the treatment of a togavirus, coronavirus or herpes virus infection in a host are provided. In particular, methods for the treatment of a togavirus, coronavirus or herpes virus infection in humans and other host animals, include administering an effective amount of a compound of the invention, or a pharmaceutically acceptable salt or prodrug thereof, optionally in a pharmaceutically acceptable carrier. Compounds of the invention disclosed herein include alkylamidophosphocholine compounds or analogues thereof or salts or prodrugs thereof. The compounds can be used singly or in combination. The methods include the use of the compounds of the invention to treat or retard the progression of clinical illness in individual infected with togavirus, coronavirus, or herpes virus, or to prevent or reduce the severity of the infection.

In particular, the methods, compounds and compositions disclosed herein are useful for the treatment of a coronavirus infection, such as a SARS-CoV infection, or a herpes virus infection, such as a Varicella-zoster virus and cytomegalovirus infection.

In a further embodiment, the methods, compounds and compositions disclosed herein are useful for the treatment of infections of enveloped, positive-stranded RNA viruses.

The compounds disclosed herein, or salts or prodrugs, can be administered or used in pharmaceutical compositions, optionally in a pharmaceutically acceptable carrier. The compounds may possess anti-viral activity and may be metabolized to a compound or compounds that exhibit anti-viral activity. Without being limited to any theory, it is possible that the effectiveness of the compounds may be due to the fact that they are phosphocholine (PC) compounds, which may provide a surfactant effect, to assist in the removal of pulmonary secretions and improve oxygenation, if administered by pulmonary administration.

In the compounds of the invention, a PC (phosphocholine) moiety is preferably incorporated into the lipid backbone to provide compounds that exhibit optimal antiviral activity.

Compounds of the invention having a chiral center can exist in and be isolated in distinct optically active or racemic forms. The present invention encompasses any racemic, optically active, or stereoisomeric form, or mixtures of such forms of a compound of the invention. Preparation of optically active forms of a compound is well known in the art, for example, by resolution of the racemic form by recrystallization techniques, by synthesis from optically active starting materials, by chiral synthesis, or by chromatographic separation using a chiral stationary phase. Determination or assessment of antiviral activity may be performed using standard tests described herein or other tests known in the art. The present invention also encompasses polymorphic forms and mixtures thereof.

In particular, the present invention provides the following:
a) a pharmaceutical composition for the treatment and/or prophylaxis of a togavirus, herpes virus, and/or coronavirus infection in a host, especially a host diagnosed as having or being at risk for such infection, comprising compound disclosed herein, or a pharmaceutically acceptable salt or prodrug thereof, optionally with a pharmaceutically acceptable carrier or diluent; and optionally with one or more other effective antiviral agents;
b) a method for the treatment of a togavirus, herpes virus and/or coronavirus infection in a host comprising administering an anti-viral effective amount of a compound disclosed herein, or a pharmaceutically acceptable salt or prodrug thereof, optionally with a pharmaceutically acceptable carrier, excipient or diluent, and optionally in combination and/or alternation with one or more other effective antiviral agents;
c) use of a compound disclosed herein, or a pharmaceutically acceptable salt or prodrug thereof, optionally with a pharmaceutically acceptable carrier or diluent, for the treatment of a togavirus, herpes virus and/or coronavirus infection in a host, optionally in combination and/or alternation with one or more other effective antiviral agents; and
d) use of a compound disclosed herein, or a pharmaceutically acceptable salt or prodrug thereof, optionally in combination and/or alternation with one or more other effective antiviral agents, and optionally with a pharmaceutically acceptable carrier or diluent, in the manufacture of a medicament for the treatment of a togavirus, herpes virus and/or coronavirus infection in a host.

All togaviruses, herpes viruses and coronaviruses are intended for inclusion within the scope of this invention. Togaviruses include, for example, rubiviruses that cause rubella and alphaviruses that cause encephalitis; human and avian coronaviruses; and the SARS coronavirus. Examples of Togaviruses that can be treated include, but are not limited, alphaviruses (such as for example Sindbis virus, Eastern/Western encephalitis viruses, Semliki Forest virus, and Ross River virus) and rubiviruses (such as for example Rubella virus).

The coronaviruses that can be treated according to this invention include, but are not limited to, Severe Acute Respiratory syndrome (SARS) coronavirus (SARS-CoV), human respiratory coronavirus (HCV-229E), porcine transmissible gastroenteritis virus (TGEV), canine coronavirus (CCV), feline enteric coronavirus (FECV), feline infectious peritonitis virus (FIPV), rabbit coronavirus (RbCV), human respiratory coronavirus (HCV-OC43), mouse hepatitis virus (MHV), sialodacryoadnavirus (SDAV), porcine hemagglutinating encephalomyelitis virus (HEV), bovine coronavirus (BCV), rabbit enitis coronavirus (RbEVC), turkey coronavirus (TCV), and avian infectious bronchitis virus (IBV).

The herpes viruses that can be treated include Varicella-zoster virus and cytomegalovirus.

Definitions

The term “alkyl” as used herein, unless otherwise specified, includes a saturated straight chain, branched, acyclic or cyclic, primary, secondary, or tertiary hydrocarbon, for example, C₁ to C₂₂, C₁-C₃, C₁-C₄, C₂-C₄, C₂-C₆, C₆-C₁₈, C₂-C₁₄, C₁-C₂₀, C₁₄-C₁₈, or C₉-C₃₀ alkyl, and specifically includes methyl, trifluoromethyl, ethyl, propyl, isopropyl, cyclopropyl, butyl, isobutyl, t-butyl, pentyl, cyclopentyl, isopentyl, neopentyl, hexyl, isohexyl, cyclohexyl, cyclohexylmethyl, 3-methylpentyl, 2,2-dimethybutyl, and 2,3-dimethylbutyl. The alkyl group can be optionally substituted with one or more moieties such as a halo (e.g. CH₂F or CF₃), acyl, amino, alkylamino, arylamino, alkoxy, aryloxy, nitro, cyano, sulfonic acid, sulfate, phosphonic acid, phosphate, or phosphonate, either unprotected, or protected as necessary, as known to those skilled in the art, for example, as taught in Greene, et al., “Protective Groups in Organic Synthesis,” John Wiley and Sons, Second Edition, 1991, hereby incorporated by reference.

As used herein, the term “C₁-C₃ alkyl” means a saturated straight chain or branched, acyclic or cyclic, primary, secondary, or tertiary hydrocarbon having from 1 to 3 carbon atoms.

As used herein, the term “C₁-C₄ alkyl” means a saturated straight chain or branched, acyclic or cyclic, primary, secondary, or tertiary hydrocarbon having from 1 to 4 carbon atoms.

As used herein, the term “C₂-C₄ alkyl” means a saturated straight chain or branched, acyclic or cyclic, primary, secondary, or tertiary hydrocarbon having from 2 to 4 carbon atoms.

As used herein, the term “C₂-C₆ alkyl” means a saturated straight chain or branched, acyclic or cyclic, primary, secondary, or tertiary hydrocarbon having from 2 to 6 carbon atoms.

As used herein, the term “C₆-C₁₈ alkyl” means a saturated straight chain or branched, acyclic or cyclic, primary, secondary, or tertiary hydrocarbon having from 6 to 18 carbon atoms.

As used herein, the term “C₂-C₁₄ alkyl” means a saturated straight chain or branched, acyclic or cyclic, primary, secondary, or tertiary hydrocarbon having from 2 to 14 carbon atoms.

As used herein, the term “C₁-C₂₂ alkyl” means a saturated straight chain or branched, acyclic or cyclic, primary, secondary, or tertiary hydrocarbon having from 1 to 22 carbon atoms.

As used herein, the term “C₁-C₂₀ alkyl” means a saturated straight chain or branched, acyclic or cyclic, primary, secondary, or tertiary hydrocarbon having from 1 to 20 carbon atoms.

As used herein, the term “C₁₄-C₁₈ alkyl” means a saturated straight chain or branched, acyclic or cyclic, primary, secondary, or tertiary hydrocarbon having from 14 to 18 carbon atoms.

As used herein, the term “C₉-C₃₀ alkyl” means a saturated straight chain or branched, acyclic or cyclic, primary, secondary, or tertiary hydrocarbon having from 9 to 30 carbon atoms.

The term “lower alkyl” as used herein, and unless otherwise specified, includes a C₁ to C₆ saturated straight chain, branched, or cyclic as in cyclopropyl, alkyl group.

As used herein, the term “alkenyl,” unless otherwise specified, includes a straight chain or branched, acyclic or cyclic, hydrocarbon having at least 2 carbon atoms and including at least one carbon-carbon double bond. Examples of alkenyl include, but are not limited to, vinyl, allyl, 1-butenyl, 2-butenyl, isobutenyl, 1-pentenyl, 2-pentenyl, 3-methyl-1-butenyl, 2-methyl-1-butenyl, 2,3-dimethyl-2-butenyl, 1-hexenyl, 2-hexenyl, 3-hexenyl, 2-heptenyl, 3-heptenyl, 1-octenyl, 2-octenyl, 3-octenyl, 1-nonenyl, 2-nonenyl, 3-nonenyl, 1-decenyl, 2-decenyl, and 3-decenyl moieties.

As used herein, the term “C₂-C₂₂ alkenyl” means a straight chain or branched, acyclic or cyclic, primary, secondary, or tertiary hydrocarbon having from 2 to 22 carbon atoms and including at least one carbon-carbon double bond.

As used herein, the term “C₂-C₂₀ alkenyl” means a straight chain or branched, acyclic or cyclic, primary, secondary, or tertiary hydrocarbon having from 2 to 20 carbon atoms and including at least one carbon-carbon double bond.

As used herein, the term “C₆-C₁₈ alkenyl” means a straight chain or branched, acyclic or cyclic, primary, secondary, or tertiary hydrocarbon having from 6 to 18 carbon atoms and including at least one carbon-carbon double bond.

As used herein, the term “C₂-C₁₄ alkenyl” means a straight chain or branched, acyclic or cyclic, primary, secondary, or tertiary hydrocarbon having from 2 to 14 carbon atoms and including at least one carbon-carbon double bond.

As used herein, the term “C₉-C₃₀ alkenyl” means a saturated straight chain or branched, acyclic or cyclic, primary, secondary, or tertiary hydrocarbon having from 9 to 30 carbon atoms and including at least one carbon-carbon double bond.

As used herein, the term “alkynyl,” unless otherwise specified, includes a straight chain or branched, acyclic hydrocarbon having at least 2 carbon atoms and including at least one carbon-carbon triple bond. Examples of alkynyl include, but are not limited to, acetylenyl, propynyl, 1-butynyl, 2-butynyl, 1-pentynyl, 2-pentynyl, 3-methyl-1-butynyl, 4-pentynyl, 1-hexynyl, 2-hecynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl, and 9-decynyl moieties.

As used herein, the term “C₂-C₂₀ alkynyl” means a straight chain or branched, acyclic primary, secondary, or tertiary hydrocarbon having from 2 to 20 carbon atoms and including at least one carbon-carbon triple bond.

As used herein, the term “C₆-C₁₈ alkynyl” means a straight chain or branched, acyclic primary, secondary, or tertiary hydrocarbon having from 6 to 18 carbon atoms and including at least one carbon-carbon triple bond.

As used herein, the term “C₂-C₁₄ alkynyl” means a straight chain or branched, acyclic primary, secondary, or tertiary hydrocarbon having from 2 to 14 carbon atoms and including at least one carbon-carbon triple bond.

As used herein, the term “C₉-C₃₀ alkynyl” means a saturated straight chain or branched, acyclic, primary, secondary, or tertiary hydrocarbon having from 9 to 30 carbon atoms and at least on carbon-carbon triple bond.

The term “aryl” as used herein and, unless otherwise specified, includes phenyl, biphenyl or naphthyl. The aryl group can optionally be substituted with one or more moieties including but not limited to halo, alkyl, hydroxyl, amino, alkylamino, arylamino, alkoxy, aryloxy, nitro, cyano, thio, alkylthio, carboxamido, carboxylate, sulfonic acid, sulfate, phosphonic acid, phosphate, or phosphonate, either unprotected or protected as necessary, as known to those skilled in the art. The aryl group is optionally substituted with one or more of alkenyl, alkynyl, —OH, —NH₂, —NHR¹, —NR¹R¹, —NH(aryl), —NH(aryl)(aryl), —O-alkyl, —O-alkenyl, —O-alkynyl, —O-aryl, nitro, cyano, —S-alkyl, —S-alkenyl, —S-alkynyl, —S—aryl, —NR¹C(O)R¹, —COOH, —SO₃H, —COOR¹, —OP(O)(OR¹)₂, —OP(O)(R¹)(OR¹), —OP(O)(R¹)₂, either unprotected or protected using a protecting group (as known to those skilled in the art, for example, as taught in Greene et al., Protective Groups in Organic Synthesis. John Wiley and Sons, 2^{nd} edition (1991)), wherein each R¹ is for example, independently hydrogen, alkyl, alkenyl, or alkynyl.

The term “halo” as used herein includes bromo, chloro, iodo and fluoro.

As used herein, the term “heterocyclic ring,” unless otherwise specified, includes a 3 to 10 membered monocyclic or bicyclic ring which is either saturated, unsaturated non-aromatic, or aromatic containing from 1 to 4 heteroatoms independently selected from nitrogen, which can be quaternized; oxygen; and sulfur, including sulfoxide and sulfone. The heterocycle ring can be attached by a nitrogen, sulfur, or carbon atom. Representative heterocycles include, but are not limited to, pyridyl, furyl, thiophenyl, pyrrolyl, oxazolyl, imidazolyl, thiazolyl, thiadiazolyl, isooxazolyl, pyrazolyl, isothiazolyl, pyridazinyl, pyrimidinyl, pyrazinyl, triazinyl, morpholinyl, pyrrolidinyl, piperidinyl, piperizinyl, hydantoinyl, valerolactamyl, oxiranyl, oxetanyl, tetrahydrofuranyl, tetrahydropyranyl, tetrahydropyridinyl, tetrahydropyrimidinyl, tetrahydrothiophenyl, tetrahydrothiopyranyl, quinolinyl, isoquinolinyl, chromonyl, coumarinyl, indolyl, indolizinyl, benzo[b[furanyl, benzo[b]thiophenyl, indazolyl, purinyl, 4H-quinolizinyl, isoquinolyl, quinolyl, phthalazinyl, naphthyridinyl, and carbazolyl.

As used herein, the term “heteroaromatic,” unless otherwise specified, includes an aromatic heterocycle ring having between 5 and 10 ring atoms, including both monocyclic and bicyclic ring systems, wherein at least one carbon atom of one or both of the rings is replaced with a heteroatom independently selected from nitrogen, oxygen, and sulfur. Representative heteroaromatics include, but are not limited to, pyridyl, furyl, benzofutanyl, thiophenyl, benzothiophenyl, quinolynyl, pyrrolyl, indolyl, oxazolyl, benzooxazolyl, imidazolyl, benzimidazolyl, thiazolyl, benzothiazolyl, isoxazolyl, pyrazolyl, isothiazolyl, pyridazinyl, pyrimidinyl, pyrazinyl, thiadiazolyl, triazinyl, cinnolinyl, phthalazinyl, and quinazolinyl.

As used herein the term “cycloalkane ring” or “cycloalkyl,” unless otherwise specified, includes a 3 to 14 membered monocyclic, bicyclic, or tricyclic hydrocarbon ring which is either saturated or unsaturated non-aromatic. Representative cycloalkane rings include, but are not limited to, cyclopropyl, cyclobutyl, cyclopentyl, cyclohexyl, cycloheptyl, cyclooctyl, indanyl, 1,2,3,4-tetrahydronaphthyl, perhydronaphthyl, 1,2,3,4-tetrahydroanthracenyl, cyclopentenyl, cyclopentadienyl, cyclohexenyl, cycloheptenyl, cyclohetadienyl, and cycloheptatrienyl.

As used herein the term “C₃-C₈ cycloalkyl” or “C₃-C₈ cycloalkane ring” includes a 3 to 8 membered monocyclic hydrocarbon ring. Representative C₃-C₈ cycloalkane rings include, but are not limited to, cyclopropyl, cyclobutyl, cyclopentyl, cyclohexyl, cycloheptyl, and cyclooctyl.

The term “host”, as used herein, refers to a unicellular or multicellular organism, in which the virus can replicate, including cell lines and animals, and preferably a human. Alternatively, the host can be carrying a part of the viral genome, whose replication or function can be altered by the compounds of the present invention. The term host refers to infected cells, cells transfected with all or part of the togavirus, herpes virus and/or coronavirus genome and animals, in particular, primates (including chimpanzees) and humans. In most animal applications of the present invention, the host is a human patient. Veterinary applications, in certain indications, however, are clearly encompassed by the present invention such as in chimpanzees.

The term “pharmaceutical salt” includes a salt that retains the desired biological activity of the parent compound and preferably does not impart undesired toxicological effects thereto. Examples of salts include, but are not limited to, (a) salts formed with cations such as sodium, potassium, NH₄⁺, magnesium, and calcium polyamines such as spermine and spermidine; (b) acid addition salts formed with inorganic acids including, but not limited to, hydrochloric acid, hydrobromic acid, sulfuric acid, phosphoric acid, and nitric acid; (c) salts formed with organic acids including, but not limited to, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, and polygalacturonic acid; and (d) salts formed from elemental anions such as chloride, bromide, and iodide.

As used herein, the term “prodrug” includes a compound that, when administered to an animal, is converted under physiological conditions to a compound of the invention.

The term “treatment” as used herein, includes an approach for obtaining beneficial or desired results including clinical results, including alleviation of symptoms, diminishment of extent of disease, stabilization (i.e., not worsening) state of disease, preventing spread of disease, preventing or reducing occurrence or recurrence of disease, delay or slowing of disease progression, and reduction of incidence of disease or symptoms. As used herein, the phrase “anti-viral effective amount” means an amount effective for treating the virus.

Compounds and Salts and Prodrugs Thereof

Compounds for the treatment of a herpes virus, togavirus and/or coronavirus infection are provided, as well as methods of use, and compositions comprising the compounds. Compounds of the invention include compounds of the structures disclosed herein as well as salts and prodrugs thereof.

In one embodiment, the compound is a compound of the general formula below: 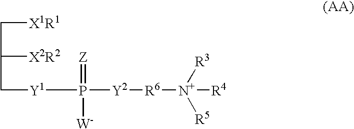
in any of its tautomeric, stereoisomeric or enantiomeric forms;
or a pharmaceutically acceptable salt or prodrug thereof, wherein:

R¹ is alkyl, alkenyl or alkynyl such as C₁-C₂₂ alkyl, alkenyl or alkynyl, or C₁-C₁₂ alkyl, alkenyl or alkynyl, optionally substituted (e.g. from 1 to 5 times) with —OH, —COOH, oxo, or amino;
R² is alkyl, such as C₁-C₂₂ alkyl, or is alkenyl or alkynyl, optionally substituted (e.g. from 1 to 5 times) with —OH, —SH, oxo, amino, —COOH, —COOR′, —NR′C(O)R″—, or —C(O)N(R′)(R″);
X¹ and X² are independently amide, carbonylamino, aminocarbonyl, ureido, ester, amine, hydrazine, —NR′—NR—, —NHC(O)—, —NR′C(O)—, —N(CH₃)C(O)—, —C(O)NH—, —C(O)NR′——C(O)N(CH₃)—, —NH—, —NR′—, —N(CH₃)—, —(C═NH)—, —(C═NR′)—, —O(C═NH)—, —O(C═NR′)—, —(C═NH)O—, —(C═NR′)O—, —S(C═NH)—, —S(C═NR′)—, —(C═NH)S—, —(C═NR′)S—, —O(C═NH)O—, —S(C═NH)O—, —O(C═NH)S—, —S(C═NH)S—, —O(C═NR′)O—, —S(C═NR′)O—, —O(C═NR′)S—, —S(C═NR′)S—, —C(O)—, —OC(O)—, —C(O)O—, —OC(O)O—, —SC(O)—, —C(O)S—, —SC(O)O—, —OC(O)S—, —SC(O)S—, —NHC(O)NH—, —NHC(O)NR—′, —N(R′)C(O)NH—, —N(R′)C(O)NR″—, —NHC(S)—, —NR′C(S)—, —N(CH₃)C(S)—, —C(S)NH—, —C(S)NR′—, —C(S)N(CH₃)—, —C(S)—, —OC(S)—, —C(S)O—, —OC(S)O—, —SC(S)—, —C(S)S—, —SC(S)O—, —OC(O)S—, —SC(S)S—, —NHC(S)NH—, —NHC(S)NR′—, —NR′C(S)NH—, —NR′C(S)NR″—, —O—, —S—, —S(O)—, —(SO₂), sulphinyl, or sulphonyl;
Y¹ and Y² are independently O, S or Se;
Z is O, S, Se, NH, or NR′;
W is O, S, NH, or NR′;
R⁶ is alkyl, alkenyl, or alkynyl, e.g., methyl or ethyl; and

R³, R⁴ and R⁵ are independently an alkyl, such as a C₁ to C₆ alkyl, preferably methyl or ethyl; or R³ and R⁴ together form a heterocyclic ring, for example having three, four, five, six or seven members, and R⁵ is an alkyl, such as a C₁ to C₆ alkyl, preferably methyl or ethyl; and

R′ and R″ are independently hydrogen, alkyl, alkenyl, alkynyl, cycloalkyl, aryl, heteroaryl, or heterocyclic.

Embodiments of compounds of Formula AA:

In one embodiment of the compound of Formula AA, X¹ is —NHC(O)—.

In another embodiment, X¹ is —N(CH₃)C(O)—.

In one embodiment, X¹ is —C(O)NH—.

In one embodiment, X¹ is —C(O)N(CH₃)—.

In one embodiment, X¹ is —NH—.

In one embodiment, X¹ is —N(CH₃)—.

In one embodiment, X² is —NHC(O)—.

In one embodiment, X² is —N(CH₃)C(O)—.

In one embodiment, X² is —C(O)_{N}(CH₃)—.

In one embodiment, X² is —S—.

In one embodiment, X² is —S(O)—.

In one embodiment, X 2 is —(SO₂)—.

In one embodiment, X² is —O—

In one embodiment, X² is —O—.

In one embodiment, X² is —NH—.

In one embodiment, X² is —N(CH₃)—.

In one embodiment, Y¹ is —O—.

In one embodiment, Y¹ is —S—.

In one embodiment, Y¹ is —Se—.

In one embodiment, Y² is —O—.

In one embodiment, Y² is —S—.

In one embodiment, Y² is —Se—.

In one embodiment, Z is —O—.

In one embodiment, Z is —S—.

In one embodiment, Z is —Se—.

In one embodiment, Z is —NH—.

In one embodiment, Z is —S—.

In one embodiment, W is —O—.

In one embodiment, W is −S—.

In one embodiment, W is —NH—.

In one embodiment, W is —NR′—.

In one embodiment, R¹ is a C₁-C₂₂ alkyl optionally substituted from 1 to 5 times with —OH, —COOH, oxo, or amino.

In one embodiment, R¹ is a C₁-C₁₂ alkyl optionally substituted from 1 to 5 times with —OH, —COOH, oxo, or amino.

In one embodiment, R¹ a C₂-C₂₂ alkenyl optionally substituted from 1 to 5 times with —OH, —COOH, oxo, or amino.

In one embodiment, R¹ is a C₂-C₁₂ alkenyl optionally substituted from 1 to 5 times with —OH, —COOH, oxo, or amino.

In one embodiment, R¹ a C₂-C₂₂ alkynyl optionally substituted from 1 to 5 times with —OH, —COOH, oxo, or amino.

In one embodiment, R¹ is a C₂-C₁₂ alkynyl optionally substituted from 1 to 5 times with —OH, —COOH, oxo, or amino.

In one embodiment, R² a C₁-C₅ alkyl optionally substituted from 1 to 5 times with —OH, —SH, oxo, amino, —N(R′)C(O)R″, —C(O)N(R′)(R″), —COOH, or —COOR′.

In one embodiment, R² a C₂-C₅ alkenyl optionally substituted from 1 to 5 times with —OH, —SH, oxo, amino, —N(R′)C(O)R″, —C(O)N(R′)(R″), —COOH, or —COOR′.

In one embodiment, R² a C₂-C₅ alkynyl optionally substituted from 1 to 5 times with —OH, —SH, oxo, amino, —N(R′)C(O)R″, —C(O)N(R′)(R″), —COOH, or —COOR′.

In one embodiment, R² C₁-C₂₂ alkyl optionally substituted from 1 to 5 times with —OH, —SH, oxo, amino, —NR′C(O)R″—, —C(O)N(R′)(R″), —COOH, or —COOR′.

In one embodiment, R² is a C₁-C₁₂ alkyl optionally substituted from 1 to 5 times with —OH, —SH, oxo, amino, —NR′C(O)R″—, —C(O)N(R′)(R″), —COOH, or —COOR′.

In one embodiment, R² is a C₂-C₂₂ alkenyl optionally substituted from 1 to 5 times with —OH, —SH, oxo, amino, —NR′C(O)R″—, —C(O)N(R′)(R″), —COOH, or —COOR′.

In one embodiment, R² is a C₂-C₁₂ alkenyl optionally substituted from 1 to 5 times with —OH, —SH, oxo, amino, —NR′C(O)R″—, —C(O)N(R′)(R″), —COOH, or —COOR′.

In one embodiment, R² is a C₂-C₂₂ alkynyl optionally substituted from 1 to 5 times with —OH, —SH, oxo, amino, —NR′C(O)R″—, —C(O)N(R′)(R″), —COOH, or —COOR′.

In one embodiment, R² is a C₂-C₁₂ alkynyl optionally substituted from 1 to 5 times with —OH, —SH, oxo, amino, —NR′C(O)R″—, —C(O)N(R′)(R″), —COOH, or —COOR′.

In one embodiment, R⁶ is a C₂-C₆ alkyl.

In one embodiment, R⁶ is —CH₂—.

In one embodiment, R⁶ is —CH₂—CH₂—.

In one embodiment, R⁶ is a C₂-C₆ alkenyl.

In one embodiment, R⁶ is a C₂-C₆ alkynyl.

In one embodiment, R³, R⁴, and R⁵ are each independently a C₁-C₆ alkyl.

In one embodiment, each R³, R⁴ and R⁵ is independently a methyl or ethyl.

In one embodiment, R³ and R⁴ together form a heterocyclic ring having between three and seven ring atoms and R⁵ is an alkyl group.

In one embodiment, R³ and R⁴ together form a heterocyclic ring having between three and seven ring atoms and R⁵ is methyl.

In one embodiment, R³ and R⁴ together form a heterocyclic ring having between three and seven ring atoms and R⁵ is ethyl.

In one embodiment, each R′ and R″ is independently a C₁-C₂₂ alkyl group.

In one embodiment, the compound is active in the treatment of a herpes virus, such as varicella zoster virus or cytomegalovirus; or a coronavirus,such as SARS-CoV.

It has been surprisingly found that phospholipids of the formula (AA) with lower alkyl chains in the R¹ and/or R² positions, and in particular the R² position, are active against viral infections, such as togavirus, herpes virus and/or coronavirus infection, and in particular an infection of varicella zoster virus, cytomegalovirus,or SARS-CoV.

In a subembodiment, compounds useful in the methods and compositions for the treatment of a togavirus, herpes virus and/or coronavirus infection, and in particular an infection of varicella zoster virus, cytomegalovirus,or SARS-CoV, are compounds of the formula below: 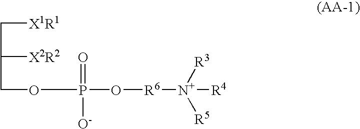
in any of its tautomeric, stereoisomeric or enantiomeric forms;
or a pharmaceutically acceptable salt and/or prodrug thereof, wherein:
X¹ is —NHC(O)—, —N(CH₃)C(O)—, —C(O)NH—, —C(O)N(CH₃)—, —NH— or —N(CH₃)—;
X² is —NHC(O)—, —N(CH₃)C(O)—, —C(O)N(CH₃)—, —S—, —S(O)—, —(SO₂)—, —O—, —NH—, or —NCH₃—;
R¹ is C₁-C₁₂ alkyl, C₂-C₁₂ alkenyl or C₂-C₁₂ alkynyl, optionally substituted from 1 to 5 times with —OH, —COOH, oxo, or amino;
R² is C₁-C₁₂ alkyl, C₂-C₁₂ alkenyl, or C₂-C₁₂ alkynyl, optionally substituted from 1 to 5 times with —OH, —COOH, oxo, or amino;
wherein optionally and at least one of R¹ or R² independently is a C₁-C₇ alkyl, C₂-C₇ alkenyl, or C₂-C₇ alkynyl;
R⁶ is C₁-C₆ alkyl, C₂-C₆ alkenyl, or C₂-C₆ alkynyl; and
R³, R⁴ and R⁵ are independently methyl or ethyl; or
R³ and R⁴ together form a heterocyclic ring having five or six members and R₅ is methyl or ethyl.

In one embodiment, one or more alkyl groups are substituted.

Embodiments of compounds of Formula AA-1:

In one embodiment of the compound of Formula AA-1, R′ is a C₁-C₅ alkyl substituted from 1 to 5 times with —OH, —COOH, oxo, or amino.

In another embodiment, R¹ is a C₂-C₅ alkenyl substituted from 1 to 5 times with —OH, —COOH, oxo, or amino.

In one embodiment, R¹ is a C₂-C₅ alkynyl substituted from 1 to 5 times with —OH, —COOH, oxo, or amino.

In one embodiment, R¹ is a C₁-C₁₂ alkyl substituted from 1 to 5 times with —OH, —COOH, oxo, or amino.

In one embodiment, R¹ is a C₂-C₁₂ alkenyl substituted from 1 to 5 times with —OH, —COOH, oxo, or amino.

In one embodiment, R¹ is a C₂-C₁₂ alkynyl substituted from 1 to 5 times with —OH, —COOH, oxo, or amino.

In one embodiment, X¹ is —NHC(O)—.

In one embodiment, X¹ is —N(CH₃)C(O)—.

In one embodiment, X¹ is —C(O)NH—.

In one embodiment, X¹ is —C(O)N(CH₃)—.

In one embodiment, X¹ is —NH—.

In one embodiment, X¹ is —N(CH₃)—.

In one embodiment, R² is a C₁-C₅ alkyl substituted from 1 to 5 times with —OH, —COOH, oxo, or amino.

In one embodiment, R² is a C₂-C₅ alkenyl substituted from 1 to 5 times with —OH, —COOH, oxo, or amino.

In one embodiment, R² is a C₂-C₅ alkynyl substituted from 1 to 5 times with —OH, —COOH, oxo, or amino.

In one embodiment, R² is a C₁-C₁₂ alkyl that is optionally substituted from 1 to 5 times with —OH, —COOH, oxo, or amino.

In one embodiment, R² is a C₂-C₁₂ alkenyl that is optionally substituted from 1 to 5 times with —OH, —COOH, oxo, or amino.

In one embodiment, R² is a C₂-C₁₂ alkynyl that is optionally substituted from 1 to 5 times with —OH, —COOH, oxo, or amino.

In one embodiment, X is —NHC(O)—.

In one embodiment, X² is —N(CH₃)C(O)—

In one embodiment, X² is —C(O)N(CH₃)—.

In one embodiment, X² is —S—.

In one embodiment, X² is —S(O)—.

In one embodiment, X² is —(SO₂)—

In one embodiment, X² is —O—.

In one embodiment, X² is —NH—.

In one embodiment, X² is —N(CH₃).—

In one embodiment, R⁶ is a C₂-C₆ alkyl.

In one embodiment, R⁶ is a C₂-C₆ alkenyl.

In one embodiment, R⁶ is a C₂-C₆ alkynyl.

In one embodiment, each R³, R⁴, and R⁵ is independently methyl or ethyl.

In one embodiment, R³ and R⁴ together form a heterocyclic ring having five or six ring atoms and R⁵ is methyl.

In one embodiment, R³ and R⁴ together form a heterocyclic ring having five or six ring atoms and R⁵ is ethyl.

In another subembodiment, compounds useful in the methods and compositions for the treatment of a togavirus, herpes virus and/or coronavirus infection, and in particular an infection of varicella zoster virus, cytomegalovirus,or SARS-CoV, are compounds of the formula below: 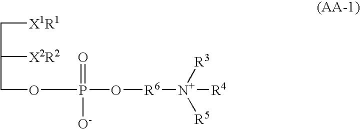
in any of its tautomeric, stereoisomeric or enantiomeric forms;
or a pharmaceutically acceptable salt and/or prodrug thereof, wherein:
X¹ is —NHC(O)—, —N(CH₃)C(O)—, —C(O)NH—, —C(O)N(CH₃)—, —NH— or —N(CH₃)—;
X² is —NHC(O)—, —N(CH₃)C(O)—, —C(O)N(CH₃)—, —S—, —S(O)—, —(SO₂)—, —O—, —NH—, or —NCH₃—;
R¹ is C₁-C₂₂ alkyl, C₂-C₂₂ alkenyl, or C₂-C₂₂ alkynyl;
R² is C₁-C₁₂ alkyl, C₂-C₁₂ alkenyl, or C₂-C₁₂ alkynyl;
wherein at least one of R¹ and R² is independently C₁-C₇ alkyl, C₂-C₇ alkenyl, or C₂-C₇ alkynyl, or at least one of R¹ and R² is independently C₂ or C₃ alkyl;
R⁶ is a C₁-C₆ alkyl, C₂-C₆ alkenyl, or C₂-C₆ alkynyl; and

R³, R⁴ and R⁵ are independently methyl or ethyl; or

R³ and R⁴ together form a heterocyclic ring having five or six members and R₅ is methyl or ethyl.

In an alternative embodiment, one or more alkyl groups disclosed herein are substituted.

Embodiments of a compound of Formula AA-1:

In one embodiment of a compound of Formula AA-1, X′ is —NHC(O)—.

In another embodiment, X¹ is —N(CH₃)C(O)—.

In one embodiment, X¹ is —C(O)NH—.

In one embodiment, X¹ is —C(O)N(CH₃)—.

In one embodiment, X¹ is —NH—.

In one embodiment, X¹ is —N(CH₃)—.

In one embodiment, X² is —NHC(O)—.

In one embodiment, X² is —N(CH₃)C(O)—

In one embodiment, X² is —C(O)N(CH₃)—.

In one embodiment, X¹ is —S—.

In one embodiment, X² is —S(O)—.

In one embodiment, X² is —(SO₂)—.

In one embodiment, X² is —O—.

In one embodiment, X² is —NH—.

In one embodiment, X² is —NCH₃.—

In one embodiment, R¹ is a C₁-C₁₂ alkyl.

In one embodiment, R¹ is a C₂-C₁₂ alkenyl.

In one embodiment, R¹ is a C₂-C₁₂ alkynyl.

In one embodiment, R² is a C₁-C₁₂ alkyl.

In one embodiment, R² is a C₂-C₁₂ alkenyl.

In one embodiment, R² is a C₂-C₁₂ alkynyl.

In one embodiment, at least one of R¹ or R² is a C₁-C₃ alkyl, C₂-C₃ alkenyl, or C₂-C₃ alkynyl.

In one embodiment, at least one of R¹ or R² is C₁-C₃ alkyl.

In one embodiment, at least one of R¹ or R² is C₂-C₃ alkenyl.

In one embodiment, at least one of R¹ or R² is C₂-C₃ alkynyl.

In one embodiment, R² is C₁-C₅ alkyl.

In one embodiment, R² is C₂-C₅ alkenyl.

In one embodiment, R² is C₂-C₅ alkynyl group.

In one embodiment, R⁶ is C₂-C₆ alkyl.

In one embodiment, R⁶ is C₂-C₆ alkenyl.

In one embodiment, R⁶ is C₂-C₆ alkynyl.

In a particular subembodiment, the compound useful composition and methods for the treatment of an infection of a togavirus, herpes virus or coronavirus,and in particular, varicella zoster virus, cytomegalovirus,or SARS-CoV, is a compound of the formula AA-1, in any of its tautomeric, stereoisomeric or enantiomeric forms; or a pharmaceutically acceptable salt and/or prodrug thereof, wherein:
X¹ is —NHC(O)—;
X¹ is S or O;
R¹ and R² are independently a C₁-C₂₂ straight chain alkyl; and at least one of R¹ and R² is independently straight chain C₁-C₅ alkyl;
R⁶ is C₂-C₆ straight chain alkyl; and
R³, R⁴ and R⁵ are independently methyl or ethyl.

In another embodiment, in the compound of formula AA-1:
X¹ is —NHC(O)—;
X² is S or O;
R¹ and R² are independently C₁-C₁₂ straight chain alkyl; and at least one of R¹ or R² is independently C₁-C₅ straight chain alkyl;
R⁶ is C₂-C₆ straight chain alkyl; and
R³, R⁴ and R⁵ are independently methyl or ethyl.

In another embodiment, in the compound of formula AA-1:
X¹ is —NHC(O)—;
X² is S or O;
R¹ is a C₁-C₂₂ straight chain alkyl, e.g., C₇-C₁;
R² is a C₁ to C₅ alkyl group, e.g., methyl or ethyl;
R⁶ is CH₂CH₂; and
R³, R⁴ and R⁵ are each methyl.

In one embodiment of the compound of formula AA-1,
X¹ is —NHC(O)—;
X² is —S— or —O—;
R¹ is a C₇-C₁₁ straight chain alkyl;
R² is a C₁-C₅ straight chain alkyl;
R⁶ is a —CH₂CH₂—; and
R³, R⁴, and R⁵ are each methyl.

In one embodiment of the compound of formula AA-1,
X¹ is —NHC(O)—;
X² is —S— or —O—;
R¹ is a C₇-C₁₁ straight chain alkyl;
R² is a methyl or ethyl;
R⁶ is a —CH₂CH₂—; and
R³, R⁴, and R⁵ are each methyl.

In one embodiment of the compound of formula AA-1,
X¹ is —NHC(O)—;
X² is —O—;
R¹ is —C₁-C₂₂ alkyl;
R² is —C₁-C₂₂ alkyl;
R⁶ is —CH₂CH₂—; and

R³, R⁴ and R⁵ are methyl.

In another embodiment, in the compound of formula AA-1:
X¹ is —NHC(O)—;
X² is 0;
R¹ is C₁-C₂₂ alkyl, e.g, —CH₃, —CH₂CH₃, —CH₂CH₂CH₃, —CH₂CH₂CH₂CH₃, —CH₂CH₂CH₂CH₂CH₃, —(CH₂)₅CH₃, —(CH₂)₆CH₃, —(CH₂)₇CH₃, —(CH₂)₈CH₃, —(CH₂)₉CH₃, —(CH₂)₁₀CH₃, —(CH₂)₁₁CH₃, —(CH₂)₁₂CH₃, or —(CH₂)₁₃CH₃;
R² is C₁-C₂₂ alkyl, e.g., —CH₃, —CH₂CH₃, —CH₂CH₂CH₃, —CH₂CH₂CH₂CH₃, —CH₂CH₂CH₂CH₂CH₃, —(CH₂)₅CH₃, —(CH₂)₆CH₃, —(CH₂)₇CH₃, —(CH₂)₈CH₃, —(CH₂)₉CH₃, —(CH₂)₁₀CH₃, —(CH₂)₁₁CH₃, —(CH₂)₁₂CH₃, or —(CH₂)₁₃CH₃;
R⁶ is CH₂CH₂; and

R³, R⁴ and R⁵ are methyl.

In one embodiment of the compound of Formula AA-1,
X¹ is —NHC(O)—;
X² is —O—;
R¹ is —(CH₂)₈CH₃, —(CH₂)₉CH₃, —(CH₂)₁₁ CH₃; —(CH₂)₁₂CH₃; —(CH₂)₁₂CH₃, or —(CH₂)₁₃CH₃;
R² is CH₃, —CH₂CH₃, —CH₂CH₂CH₃, —CH₂CH₂CH₂CH₃, —CH₂CH₂CH₂CH₂CH₃, —(CH₂)₅CH₃, —(CH₂)₆CH₃, or —(CH₂)₇CH₃;
R⁶ is —CH₂CH₂—; and

R³, R⁴ and R⁵ are methyl.

In one embodiment of the compound of Formula AA-1,
X¹ is —NHC(O)—;
X² is —O—;
R¹ is —(CH₂)₅CH₃, —(CH₂)₆CH₃, —(CH₂)₇CH₃, —(CH₂)₈CH₃, —(CH₂)₉CH₃, —(CH₂)₁₀CH₃, —(CH₂)₁₁,CH₃, or —(CH₂)₁₂CH₃;
R² is —(CH₂)₆CH₃, —(CH₂)₇CH₃, —(CH₂)₈CH₃, —(CH₂)₉CH₃, —(CH₂)₁₀CH₃, —(CH₂)₁₁CH₃, —(CH₂)₁₂CH₃, or —(CH₂)₁₃CH₃;
R⁶ is —CH₂CH₂—; and

R³, R⁴ and R⁵ are methyl.

In a specific embodiment, compounds useful in methods and compositions for the treatment of an infection of a togavirus, herpes virus and/or coronavirus infection, and in particular varicella zoster virus, cytomegalovirus,or SARS-CoV, are provided, wherein the compound is: 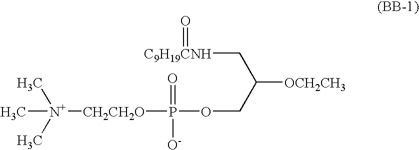
in any of its tautomeric, stereoisomeric or enantiomeric forms; or a pharmaceutically acceptable salt and/or prodrug thereof.

In another specific embodiment, a compound for use in methods and compositions for the treatment of a togavirus, herpes virus and/or coronavirus infection, and in particular a varicella zoster virus, cytomegalovirus,or SARS-CoV infection, is provided having the structure: 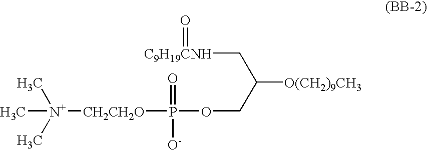
in any of its tautomeric, stereoisomeric or enantiomeric forms; or a pharmaceutically acceptable salt and/or prodrug thereof.

In another specific embodiment, the compound useful in methods and compositions for the treatment of a togavirus, herpes virus and/or coronavirus infection is the compound: 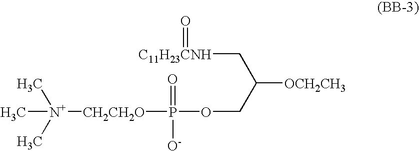
in any of its tautomeric, stereoisomeric or enantiomeric forms; or a pharmaceutically acceptable salt and/or prodrug thereof.

In a particular embodiment, the compound BB-3 is useful in methods and compositions for the treatment of a herpes virus, such as varicella zoster virus or cytomegalovirus, or for the treatment of a coronavirus,such as SARS-CoV.

In another specific embodiment, the compound for the treatment of a togavirus, herpes virus and/or coronavirus infection is the compound: 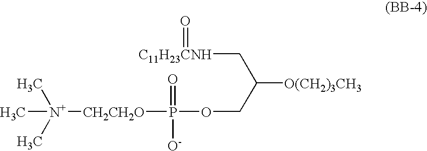
in any of its tautomeric, stereoisomeric or enantiomeric forms; or a pharmaceutically acceptable salt and/or prodrug thereof.

The compound BB-4 can be used for example in methods and compositions for the treatment of a herpes virus, such as varicella zoster virus or cytomegalovirus,or for the treatment of a coronavirus,such as SARS-CoV.

In another subembodiment, the compounds which can be used in methods and compositions for the treatment of a togavirus, herpes virus and/or coronavirus infection are the phospholipids compounds disclosed in PCT publication WO 91/09602 (Boehringer Mannheim), which is herein disclosed by reference, and in particular is a phospholipid of the formula: 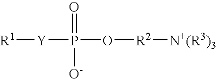
in any of its tautomeric, stereoisomeric or enantiomeric forms;
or a pharmaceutically acceptable salt or prodrug thereof, wherein:
R¹ is a straight-chain or branched, saturated or unsaturated aliphatic residue, in particular an alkyl residue, with 9 to 30 carbon atoms, which can also be part of a C₅-C₇ cycloalkane ring and may be substituted with one or more hydroxy, halogen, nitrile, a C₅-C₇ cycloalkyl, phenyl, C₁-C₂₀ alkoxy carbonyl, C₁-C₂₀ alkyl carbonyl, C₁-C₂₀ alkyl carbamoyl, C₁-C₂₀ alkyl mercapto, C₁-C₂₀ alkane sulphinyl, C₁-C₂₀ alkane sulphonyl, C₁-C₂₀ acyl amino groups or by C₁-C₂₀ alkoxy which in turn can be substituted by phenyl, C₁-C₂₀ alkyl mercapto, C₁-C₂₀ alkane sulphinyl, C₁-C₂₀ alkane sulphonyl, C₁-C₂₀ acyl amino, C₁-C₂₀ alkoxy carbonyl, nitrile, hydroxy, C₁-C₂₀ alkoxy or C₁-C₂₀ alkyl carbamoyl;
R² is a straight-chain or branched alkylene chain with 2 to 6, preferably 2 to 4, carbon atoms,
R³ is hydrogen or a C₁-C₆ alkyl group, and
Y is an oxygen or a sulphur atom.

In yet another subembodiment, compounds that can be used in methods and compositions for the treatment of a togavirus, herpes virus and/or coronavirus infection are the phospholipid compounds disclosed in WO 91/05558 (Boehringer Mannheim), which is herein disclosed by reference, and in particular a phospholipid of the formula: 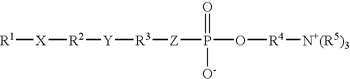
in any of its tautomeric, stereoisomeric or enantiomeric forms;
or a pharmaceutically acceptable salt or prodrug thereof, wherein:
X is a valence bond, an oxygen atom or sulphur atom, a sulphinyl, sulphonyl, carbonyl, aminocarbonyl, carbonylamino or ureido (—NH—CO—NH—) group or a C₃-C₈ cycloalkylene or phenylene residue,
Y is an oxygen atom or the groups —O—CO—O—, —O—CO—NH—, —O—CS—NH—,
R¹ is a hydrogen atom, a straight-chain or branched, saturated or unsaturated alkyl residue with 1-18 or 2-18 carbon atoms, respectively, which may be substituted one or more times by phenyl, halogen, C₁-C₄ alkoxy, C₁-C₄ alkylmercapto, C₁-C₄ alkoxycarbonyl, C₁-C₄ alkane sulphinyl or C₁-C₄ alkane sulphonyl groups,
R² is a straight or branched, saturated or unsaturated alkylene chain with 1-18 or 2-18 carbon atoms, respectively, which may be substituted one or more times by halogen, phenyl, C₁-C₄ alkoxy, C₁-C₄ alkoxycarbonyl, C₁-C₄ alkylmercapto, C₁-C₄ alkane sulphinyl or C₁-C₄ alkane sulphonyl groups,
R³ is a straight or branched, saturated or unsaturated alkylene chain with 2-8 carbon atoms which can also be substituted,
R⁴ is a straight or branched alkylene chain with 2-5 carbon atoms,
R⁵ is hydrogen or a C₁-C₆ alkyl group and
Z is oxygen or sulphur.

In another subembodiment, compounds that can be used in methods and compositions for the treatment of a togavirus, herpes virus and/or coronavirus infection are the phospholipid compounds disclosed in U.S. Pat. No. 4,444,766 (Boehringer Mannheim), which is herein disclosed by reference, and in particular a phospholipid of the formula: 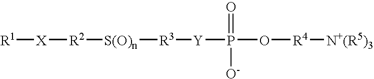
in any of its tautomeric, stereoisomeric or enantiomeric forms;
or a pharmaceutically acceptable salt or prodrug thereof, wherein:
X is a valency bond, an oxygen or sulphur atom, a sulphinyl or sulphonyl group, an aminocarbonyl, carbonylamino or ureido group or a cycloalkylene radical or a phenylene radical,
Y is an oxygen or sulphur atom,
R¹ is a hydrogen atom, a straight-chained or branched, saturated or unsaturated aliphatic hydrocarbon radical containing up to 18 carbon atoms, which is optionally substituted one or more times by aryl, halogen, lower alkoxy, alkylthio, alkoxycarbonyl, alkanesulphinyl or alkanesulphonyl,
R² is a straight-chained or branched, saturated or unsaturated aliphatic hydrocarbon chain containing up to 18 carbon atoms, which is optionally substituted one or more times by halogen, aryl, lower alkoxy, alkoxycarbonyl, alkylthio, alkanesulphinyl or alkanesulphonyl,
R³ is a straight-chained or branched, saturated or unsaturated aliphatic hydrocarbon chain containing 2 to 8 carbon atoms, which can also be part of a cycloalkane ring and which is optionally substituted one or more times by hydroxy, halogen, nitrile, cycloalkyl, phenyl, alkoxycarbonyl, optionally alkylated carbamoyl, alkylthio, alkanesulphinyl, alkanesulphonyl, optionally acylated amino or by alkoxy which, in turn, can be substituted by aryl, alkylthio, alkanesulphinyl, alkanesulphonyl, optionally acylated amino, alkoxycarbonyl, nitrile, hydroxyl, alkoxy or optionally alkylated carbamoyl,
R⁴ is a straight-chained or branched alkylene chain containing 2 to 4 carbon atoms,
R⁵ is a hydrogen atom or a lower alkyl radical and n is 0, 1 or 2.

Also provided are compounds, or pharmaceutically acceptable salts or prodrugs thereof for the treatment of a coronovirus, herpes virus or togavirus infection, as well as methods of use and pharmaceutical compositions comprising the compounds, wherein the compounds are of Formula I: 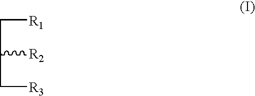
wherein:
R¹ is —NHC(O)Y, where Y is C₁-C₂₂ alkyl, C₂-C₂₂ alkenyl, or C₂-C₂₂ alkynyl;
R₂ is —OX, where X is C₁-C₂₂ alkyl, C₂-C₂₂ alkenyl, or C₂-C₂₂ alkynyl; and
R₃ is phosphocholine (OPO₃⁻CH₂CH₂N⁺(CH₃)₃).

Embodiments of compounds of Formula I:

In one embodiment of the compound of Formula I, Y is C₁-C₂₂ alkyl.

In another embodiment, Y is C₂-C₂₂ alkenyl.

In one embodiment, Y is C₂-C₂₂ alkynyl.

In one embodiment, X is C₁-C₂₂ alkyl.

In one embodiment, X is C₂-C₂₂ alkenyl.

In one embodiment, X is C₂-C₂₂ alkynyl.

In one embodiment, X is a C₁-C₅ alkyl.

In one embodiment, X is C₂-C₅ alkenyl.

In one embodiment, X is C₂-C₅ alkynyl.

In one embodiment, X is —C₂H₅ or —C₁₀H₂₁.

In one embodiment, X is —C₂H₅.

In one embodiment, Y is C₉H₁₉ or C₁₁H₂₃. Optionally, X is C₂H₅ or C₂H₅ or C₁₀H₂₁.

In one embodiment, Y is —C₁₁ H₂₃, X is —C₂H₅, and R₃ is phosphocholine.

In another embodiment, Y is —C₉H₁₉, X is —C₂H₅, and R₃ is phosphocholine.

In yet another embodiment, Y is —C₉H₁₉, X is —C₁₀H₂₁, and R₃ is phosphocholine.

The compounds, useful in methods and compositions for the treatment of a togavirus, herpes virus and/or coronavirus infection, also include compounds, or pharmaceutically acceptable salts or prodrugs thereof, of Formula II: 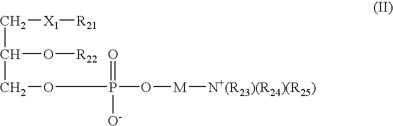
wherein:
M is a C₂-C₄ alkyl;
X₁ is —S—, —O—, —NH—, or —NHC(O)—;
R₂₁ is a C₁-C₂₀ straight chain alkyl, C₂-C₂₀ straight chain alkylene containing not more than four double bonds, or aryl;
R₂₂ is H, C₁-C₂₀ straight chain alkyl or C₂-C₂₀ straight chain alkylene containing not more than four double bonds, or aryl; and

R₂₃, R₂₄, and R₂₅ are each independently either hydrogen, methyl, ethyl, propyl, or isopropyl.

Embodiments of compounds of Formula II:

In one embodiment, M is —CH₂CH₂—.

In one embodiment, M is —CH₂CH₂CH₂—.

In one embodiment, M is —CH₂CH₂CH₂CH₂—.

In one embodiment, M is —CH₂CH(CH₃)—

In one embodiment, M is —CH(CH₃)CH₂—.

In one embodiment, M is —CH(CH₃)CH₂CH₂—.

In one embodiment, M is —CH₂CH(CH₃)CH₂—.

In one embodiment, M is —CH₂CH₂CH(CH₃)—.

In one embodiment, M is —C(CH₃)₂—.

In one embodiment, M is —CH₂C(CH₃)₂—.

In one embodiment, M is —C(CH₃)₂CH₂—.

In one embodiment, X₁ is —S—.

In one embodiment, X₁ is —O—.

In one embodiment, X₁ is —NH—.

In one embodiment, X₁ is —NHC(O)—.

In one embodiment, R₂₁ is a C₂-C₂₀ straight chain alkyl.

In one embodiment, R₂₁ is a straight chain C₂-C₂₀ alkylene containing not more than four double bonds.

In one embodiment, R₂₁ is aryl.

In one embodiment, R₂₂ is hydrogen.

In one embodiment, R₂₂ is methyl.

In one embodiment, R₂₂ is ethyl.

In one embodiment, R₂₂ is C₁-C₅ alkyl.

In one embodiment, R₂₂ is C₂-C₅ alkenyl.

In one embodiment, R₂₂ is C₂-C₅ alkynyl.

In one embodiment, R₂₃, R₂₄, and R₂₅ are methyl.

In one embodiment of Formula II:
M is CH₂CH₂;X₁ is NHC(O);
R₂₁ is C₁₆-C₁₈ linear alkyl or alkenyl containing not more than one double bond;
R₂₂ is hydrogen, methyl, or ethyl; and R₂₃, R₂₄, and R₂₅ are each independently hydrogen or methyl, preferably methyl.

In another embodiment of Formula II,
M is CH₂CH₂;
X₁ is NHC(O);
R₂₁ is a C₁₆-C₁₈ straight chain alkyl or C₁₆-C₁₈ straight chain alkenyl containing not more than one double bond;
R₂₂ is hydrogen, methyl or ethyl;
R₂₃, R₂₄ and R₂₅ are each independently CH₃.

In one embodiment of Formula II,
M is —CH₂CH₂—;
X₁ is —NHC(O)—;
R₂₁ is —C₁₁ H₂₃ or —C₉H₁₉;
R₂₂ is —C₂H₅ or —C₁₀H₂₁; and
R₂₃, R₂₄ and R₂₅ are each methyl.

The compounds useful in methods and compositions for the treatment of a togavirus, herpes virus and/or coronavirus also include compounds, or pharmaceutically acceptable salts or prodrugs thereof, of Formula III: 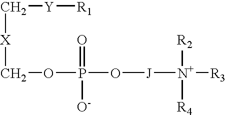
wherein:
Y is —S—, —O—, —NH—, —N(CH₃)—, —NHC(O)—, or —N(CH₃)C(O)—;
R₁ is C₁₄-C₁₈ alkyl, C₁₄-C₁₈ alkenyl, C₁₄-C₁₈ alkynyl, or aryl;
X is a covalent bond or methylene that is optionally substituted with a hydroxyl, C₁-C₂₀ alkyl, —O—(C₁-C₂₀ alkyl), —S—(C₁-C₂₀ alkyl), —C(O)N(C₁-C₂₀ alkyl), C₂-C₂₀ alkenyl, —O—(C₂-C₂₀ alkenyl), —S—(C₂-C₂₀ alkenyl), —C(O)N(C₂-C₂₀ alkenyl), C₂-C₂₀ alkynyl, —O—(C₂-C₂₀ alkynyl), —S—(C₂-C₂₀ alkynyl) or —C(O)N(C₂-C₂₀ alkynyl);
J is a C₁-C₄ alkyl that is optionally substituted one to three times with methyl or ethyl; and
R₂, R₃, and R⁴ are independently hydrogen or C₁-C₃ alkyl.

Embodiments of a compound of Formula III:

In one embodiment, Y is —S—.

In one embodiment, Y is —O—.

In one embodiment, Y is —NH—.

In one embodiment, Y is —N(CH₃)—.

In one embodiment, Y is —NHC(O)—.

In one embodiment, Y is —N(CH₃)C(O)—.

In one embodiment, R₁ is C₁₄-C₁₈ alkyl.

In one embodiment, R₁ is C₁₄-C₁₈ alkenyl.

In one embodiment, R₁ is C₁₄-C₁₈ alkynyl.

In one embodiment, R₁ is aryl.

In one embodiment, X is a covalent bond.

In one embodiment, X is a methylene that is optionally substituted with a hydroxyl, C₁-C₂₀ alkyl, —O—(C₁-C₂₀ alkyl), —S—(C₁-C₂₀ alkyl), —C(O)N(C₁-C₂₀ alkyl), C₂-C₂₀ alkenyl, —O—(C₂-C₂₀ alkenyl), —S—(C₂-C₂₀ alkenyl), —C(O)N(C₂-C₂₀ alkenyl), C₂-C₂₀ alkynyl, —O—(C₂-C₂₀ alkynyl), —S—(C₂-C₂₀ alkynyl) or —C(O)N(C₂-C₂₀ alkynyl).

In one embodiment, X is a methylene that is optionally substituted with hydroxyl, C₁-C₅ alkyl, —O—(C₁-C₅ alkyl), —S—(C₁-C₅ alkyl), —C(O)N(C₁-C₅ alkyl), C₂-C₅ alkenyl, —O—(C₂-C₅ alkenyl), —S—(C₂-C₅ alkenyl), —C(O)N(C₂-C₅ alkenyl), C₂-C₅ alkynyl, —O—(C₂-C₅ alkynyl), —S—(C₂-C₅ alkynyl) or —C(O)N(C₂-C₅ alkynyl).

In one embodiment R₂, R₃, and R⁴ are methyl.

In one embodiment, J is —CH₂CH₂—.

In one embodiment of Formula III,

Y is —NHC(O)—;
R₁ is —C₁₁ H₂₃ or —CH₉H₁₉;
X is —CH(OC₂H₅) or CH(OC₁₀H₂₁);
J is —CH₂CH₂—; and

R₂, R₃, and R⁴ are each methyl.

Compounds useful in the methods and compositions for the treatment of a togavirus, herpes virus and/or coronavirus infection further include compounds, or pharmaceutically acceptable salts or prodrugs thereof, of Formula IV: 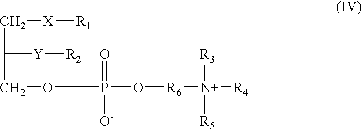
wherein:
R₁ is a C₆-C₁₈ alkyl, C₆-C₁₈ alkenyl, or C₆-C₁₈ alkynyl that is optionally substituted from 1 to 5 times with —OH, —COOH, oxo, amino, or aryl;
X is —NHC(O)—, —N(CH₃)C(O)—, —C(O)NH—, —C(O)N(CH₃)—, —S—, —S(O)—, —(SO₂)—, —O—NH—, or —N(CH₃)—;
R₂ is a C₁-C₁₄ alkyl, C₂-C₁₄ alkenyl, or C₂-C₁₄ alkynyl that is optionally substituted from 1 to 5 times with —OH, —COOH, oxo, amino, or aryl;
Y is —NHC(O)—, —N(CH₃)C(O)—, —C(O)NH—, —C(O)N(CH₃)—, —S—, —S(O)—, —(SO₂)—, —O—, —NH—, —N(CH₃)—, or —OC(O)—;
R⁶ is a C₂-C₆ alkyl; C₂-C₆ alkenyl, or C₂-C₆ alkynyl; and
R₃, R₄, and R₅ are independently methyl or ethyl, or R₃ and R⁴ together form an aliphatic or heterocyclic ring having five or six ring atoms and R₅ is methyl or ethyl.

In one embodiment of the compound of Formula IV, R₁ is a C₆-C₁₈ alkyl that is optionally substituted from 1 to 5 times with —OH, —COOH, oxo, amino.

In one embodiment, R₁ is a C₆ to C₁₈ alkenyl that is optionally substituted from 1 to 5 times with —OH, —COOH, oxo, amino.

In one embodiment, R₁ is a C₆-C₁₈ alkynyl that is optionally substituted from 1 to 5 times with —OH, —COOH, oxo, amino.

Other embodiments of a compound of Formula IV:

In one embodiment, R₁ is aryl.

In one embodiment, X is —NHC(O)—.

In one embodiment, X is —N(CH₃)C(O)—.

In one embodiment, X is —C(O)NH—.

In one embodiment, X is —C(O)N(CH₃)—.

In one embodiment, X is —S—.

In one embodiment, X is —S(O)—.

In one embodiment, X is —(SO₂)—.

In one embodiment, X is —O—.

In one embodiment, X is —NH—.

In one embodiment, X is —N(CH₃)—.

In one embodiment, R₂ is a C₁-C₁₄ alkyl that is optionally substituted from 1 to 5 times with —OH, —COOH, oxo, amino.

In one embodiment, R₂ is a C₂-C₁₄ alkenyl that is optionally substituted from 1 to 5 times with —OH, —COOH, oxo, amino.

In one embodiment, R₂ is a C₂-C₁₄ alkynyl that is optionally substituted from 1 to 5 times with —OH, —COOH, oxo, amino.

In one embodiment, R₂ is a C₁-C₅ alkyl that is optionally substituted from 1 to 5 times with —OH, —COOH, oxo, amino.

In one embodiment, R₂ is a C₂-C₅ alkenyl that is optionally substituted from 1 to 5 times with —OH, —COOH, oxo, amino.

In one embodiment, R₂ is a C₂-C₅ alkynyl that is optionally substituted from 1 to 5 times with —OH, —COOH, oxo, amino.

In one embodiment, R₂ is aryl.

In one embodiment, Y is —NHC(O)—.

In one embodiment, Y is —N(CH₃)C(O)—.

In one embodiment, Y is —C(O)NH—.

In one embodiment, Y is —C(O)N(CH₃)—.

In one embodiment, Y is —S—.

In one embodiment, Y is —S(O)—.

In one embodiment, Y is —(SO₂)—.

In one embodiment, Y is —O—.

In one embodiment, Y is —NH—.

In one embodiment, Y is —N(CH₃)—.

In one embodiment, Y is —OC(O)—.

In one embodiment, R₆ is a C₂-C₆ alkyl.

In one embodiment, R₆ is a C₂-C₆ alkenyl.

In one embodiment, R₆ is a C₂-C₆ alkynyl.

In one embodiment, R₃, R₄, and R₅ are methyl.

In one embodiment, R₃, R₄, and R₅ are ethyl.

In one embodiment, R₃ and R₄, together form an aliphatic or heterocyclic ring having five or six ring atoms and R₅ is methyl or ethyl.

In one embodiment of Formula IV,
X is —NHC(O)—;
R₁ is —C₁₁C₂₃ or —C₉H₁₉;
Y is —O—;
R₂ is —C₂H₅ or —C₁₀H₂₁;
R₆ is —CH₂CH₂—; and

R₃, R₄, and R₅ are each methyl.

Exemplary compounds for the treatment of a togovirus, coronovirus or herpes virus infection include: 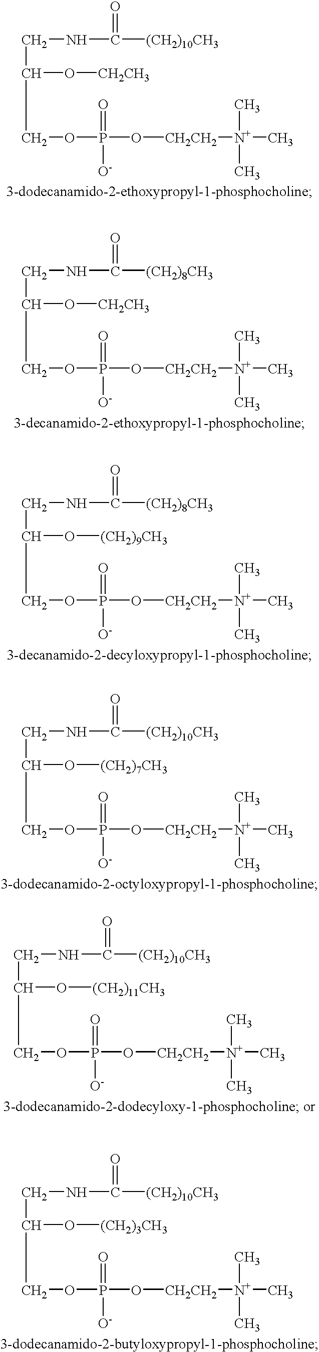
or a combination thereof.

In addition, exemplary compounds include any of the compounds disclosed in Ouyang et al., J. Med. Chem., 45:2857-2866 (2002), the disclosure of which is hereby incorporated by reference. Other compounds that may be used, alone or in combination, for the treatment of togavirus, herpes virus, and/or coronavirus infections, as disclosed herein, include compounds disclosed in U.S. Pat. No. 5,614,548, U.S. Pat. No. 5,962,437, and U.S. Pat. No. 5,770,584, the disclosures of which are hereby incorporated by reference.

Methods of Synthesis of Compounds

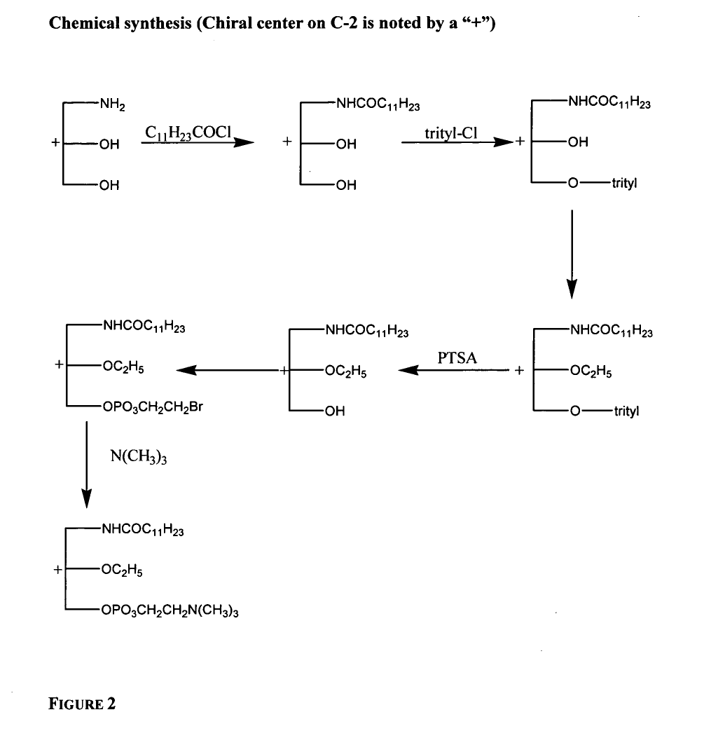

The compounds may be synthesized by methods available in the art. Alkylamido-phosphocholines may be prepared according to the method disclosed in Ouyang et al., J. Med. Chem. 45(13): 2857-2866 (2002), U.S. Pat. No. 5,614,548, U.S. Pat. No. 5,962,437, or U.S. Pat. No. 5,770,584, or as described in FIG. 2 and Example 3. 3-alkylamido-2-alkoxypropylphosphocholine is obtained by reacting commercially available 3-amino-1,2-propanediol with the appropriate acid chloride or anhydride. The primary alcohol is protected, and the secondary alcohol is alkylated with an alkyl bromide. The primary alcohol is deprotected, and then reacted with 2-bromoethyl dichlorophosphate and trimethylamine, to obtain the 3-alkylamido-2-alkoxypropylphosphocholine compound.

The compounds of the invention can also be prepared using the methods disclosed in, Morris-Natschke et al., “Synthesis Of Phosphocholine And Quaternary Amine Ether Lipids And Evaluation Of In Vitro Antineoplastic Activity,” Journal of Medicinal Chemistry 1993; 36:2018-2025; Piantadosi et al., “Synthesis and evaluation of novel ether lipid nucleoside conjugates for anti-HIV-1 activity,” Journal of Medicinal Chemistry 1991; 34:1408-1414; Kucera et al., “Synthesis And Evaluation Of A Novel Synthetic Phosphocholine Lipid-AZT Conjugate That Double-Targets Wild-Type And Drug Resistant Variants Of HIV,” Nucleosides, Nucleotides, and Nucleic Acids 2004; 23:385-399; Meyer K L, Marasco et al., “In Vitro Evaluation of Phosphocholine and Quaternary Ammonium Containing Lipids as Novel Anti-HIV Agents,” Journal of Medicinal Chemistry 1991; 34:1377-1383; and Morris-Natschke et al., “Synthesis Of Sulfur Analogues Of Alkyl Lysophospholipid And Neoplastic Cell Growth Inhibitory Properties,” Journal of Medicinal Chemistry 1986; 29:2114-2117.

Other compounds which may be used, alone or in combination, for the treatment of viral infections, as disclosed herein, and the synthesis thereof, are disclosed in U.S. Pat. No. 5,614,548, U.S. Pat. No. 5,962,437, and U.S. Pat. No. 5,770,584, which are hereby incorporated by reference.

Pharmaceutically Acceptable Salts and Prodrugs

The term “pharmaceutically acceptable salt or prodrug” includes any pharmaceutically acceptable form (such as an ester, amide, salt of an ester, salt of an amide or a related group) of the compounds described herein that, upon administration to a patient, provides the active compound.

The pharmaceutically acceptable salts preferably retain the desired biological activity of the herein-identified compounds and exhibit minimal undesired toxicological effects. Pharmaceutically acceptable salts include those derived from pharmaceutically acceptable inorganic or organic acids and bases. Non-limiting examples of suitable salts include those derived from inorganic acids such as, hydrochloric acid, hydrobromic acid, sulfuric acid, phosphoric acid, nitric acid, bicarbonic acid, carbonic acid and the like, and salts formed with organic acids such as amino acid residue, acetic acid, oxalic acid, tartaric acid, succinic acid, malic acid, malonic acid, ascorbic acid, citric acid, benzoic acid, tannic acid, palmoic acid, alginic acid, polyglutamic acid, tosic acid, methanesulfonic acid, naphthalenesulfonic acid, naphthalenedisulfonic acid, α-ketoglutaric acid, α-glycerophosphoric acid and polygalacturonic acid. Suitable salts include those derived from alkali metals such as lithium, potassium and sodium, alkaline earth metals such as calcium and magnesium, among numerous other acids well known in the pharmaceutical art. Other suitable salts include those derived from other metal cations such as zinc, bismuth, barium, aluminum, copper, and the like, or with a cation formed from an amine, such as ammonia, N,N-dibenzylethylene-diamine, D-glucosamine, tetraethylammonium, or ethylenediamine. Further, suitable salts include those derived from a combinations of acids and bases, for example, a zinc tannate salt or the like.

Further examples of salts are salts formed with organic acids such as fumaric, gluconic, citric, methanesulfonic, p-toluenesulfonic, napthalenesulfonic, and polygalacturonic acids, and the like; salts formed from elemental anions such as chloride, bromide, and iodide; salts formed from metal hydroxides, for example, sodium hydroxide, potassium hydroxide, calcium hydroxide, lithium hydroxide, and magnesium hydroxide; salts formed from metal carbonates, for example, sodium carbonate, potassium carbonate, calcium carbonate, and magnesium carbonate; salts formed from metal bicarbonates, for example, sodium bicarbonate and potassium bicarbonate; salts formed from metal sulfates, for example, sodium sulfate and potassium sulfate; and salts formed from metal nitrates, for example, sodium nitrate and potassium nitrate.

Pharmaceutically acceptable and non-pharmaceutically acceptable salts may be prepared using procedures well known in the art, for example, by reacting a sufficiently basic compound such as an amine with a suitable acid comprising a physiologically acceptable anion. Alkali metal (for example, sodium, potassium, or lithium) or alkaline earth metal (for example, calcium) salts of carboxylic acids can also be made.

Pharmaceutically acceptable prodrugs include compounds that are metabolized, for example, hydrolyzed or oxidized, in the host to form the compound of the present invention. Typical examples of prodrugs include compounds that have biologically labile protecting groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, dephosphorylated to produce the active compound. The compounds of this invention possess antiviral activity against a togavirus and/or coronavirus or are metabolized to a compound that exhibits such activity.

Any of the compounds described herein can be administered as a prodrug to increase the activity, bioavailability, stability or otherwise alter the properties of the phospholipid. A number of prodrug ligands are known. In general, alkylation, acylation or other lipophilic modification of the compound can increase the stability of the compound.

Pharmaceutical Compositions and Administration

Hosts, including humans can be treated by administering to the patient an effective amount of the active compound or a pharmaceutically acceptable prodrug or salt thereof in the presence of a pharmaceutically acceptable carrier or diluent. The active materials can be administered by any appropriate route, for example, orally, parenterally, intravenously, intradermally, subcutaneously, or topically, in liquid or solid form.

An optional dose of the compound for treatment of a togavirus, herpes virus and/or coronavirus infection is about 1 to 50 mg/kg, preferably 1 to 20 mg/kg, of body weight per day, more generally 0.1 to about 100 mg per kilogram body weight of the recipient per day. The effective dosage range of the pharmaceutically acceptable salts and prodrugs can be calculated based on the weight of the parent nucleoside to be delivered. If the salt or prodrug exhibits activity in itself, the effective dosage can be estimated as above using the weight of the salt or prodrug, or by other means known to those skilled in the art.

The compound is conveniently administered in unit any suitable dosage form, including but not limited to one containing 7 to 3000 mg, preferably 70 to 1400 mg of active ingredient per unit dosage form. An oral dosage of 50-1000 mg is optional.

Optionally, the active ingredient should be administered to achieve peak plasma concentrations of the active compound of from about 0.2 to 70 μM, e.g., about 1.0 to 10 μM. This may be achieved, for example, by the intravenous injection of a 0.1 to 5% solution of the active ingredient, optionally in saline, or administered as a bolus of the active ingredient.

The concentration of active compound in the drug composition will depend on absorption, inactivation and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient may be administered at once, or may be divided into a number of smaller doses to be administered at varying intervals of time.

The active compound can be administered in a pharmaceutically acceptable carrier available in the art, and can be administered by a chosen route of administration. Pharmaceutical compositions can be prepared, packaged, or sold in a variety of formulations which can be suitable for one or more routes of administration such as, for example, oral, intravenous, intramuscular, topical, subcutaneous, rectal, vaginal, parenteral, pulmonary, intranasal, buccal, ophthalmic, or another route of administration. The active materials can be administered in liquid or solid form. Other contemplated formulations include projected nanoparticles, liposomal preparations, resealed erythrocytes containing the active ingredient, and immunologically-based formulations.

Although the descriptions of pharmaceutical compositions provided herein are principally directed to pharmaceutical compositions which are suitable for ethical administration to humans, it will be understood by the skilled artisan that such compositions are generally suitable for administration to hosts of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates and mammals including commercially relevant mammals such as cattle, pigs, horses, sheep, cats, and dogs.

Thus, the present compounds may be systemically administered (e.g., orally) in combination with a pharmaceutically acceptable vehicle such as an inert diluent or an assimilable edible carrier. They can be enclosed in hard or soft shell gelatin capsules, compressed into tablets, or incorporated directly into the food of the patient's diet. For oral therapeutic administration, the active compound can be combined with one or more excipients and used in the form of ingestible tablets, buccal tablets, troches, capsules, elixirs, suspensions, syrups, wafers, and the like.

The concentration of active compound in the drug composition will depend on absorption, inactivation, and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient may be administered at once, or may be divided into a number of smaller doses to be administered at varying intervals of time. Pharmaceutically compatible binding agents, and/or adjuvant materials may also be included as part of the composition.

Such compositions and preparations can contain at least 0.1% (w/w) of active compound. The percentage of the compositions and preparations can, of course, be varied, for example from about 0.1% to nearly 100% of the weight of a given unit dosage form. The amount of active compound in such therapeutically useful compositions is such that an effective dosage level will be obtained upon administration.

The tablets, troches, pills, capsules, and the like may also contain one or more of the following: binders, such as microcrystalline cellulose, gum tragacanth, acacia, corn starch, or gelatin; excipients, such as dicalcium phosphate, starch or lactose; a disintegrating agent, such as corn starch, potato starch, alginic acid, primogel, and the like; a lubricant, such as magnesium stearate or Sterotes; a glidant, such as colloidal silicon dixoide; a sweetening agent, such as sucrose, fructose, lactose, saccharin, or aspartame; a flavoring agent such as peppermint, methylsalicylate, oil of wintergreen, or cherry flavoring; and a peptide antiviral agent, such as envuvirtide (Fuzeon™). When the unit dosage form is a capsule, it can contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules can be coated with gelatin, wax, shellac, sugar, and the like. A syrup or elixir can contain the active compound, sucrose or fructose as a sweetening agent, methyl and propylparabens as preservatives, a dye, and flavoring such as cherry or orange flavor. Of course, any material used in preparing a unit dosage form should be pharmaceutically acceptable and substantially non-toxic in the amounts employed. In addition, the active compound may be incorporated into sustained-release preparations and devices.

The compound or a pharmaceutically acceptable derivative or salt thereof may also be mixed with other active materials that do not impair the desired action, or with materials that supplement the desired action, such as antibiotics, antifungals, antiinflammatories, protease inhibitors, or other nucleoside or normucleoside antiviral agents. Solutions or suspensions used for parenteral, intradermal, subcutaneous, or topical application may include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation may be enclosed in ampoules, disposable syringes or multiple dose vials made of glass or plastic.

In one embodiment, the active compounds are prepared with carriers that will protect the compound against rapid elimination from the body, such as a controlled release formulation, including implants and microencapsulated delivery systems. Biodegradable, biocompatible polymers may be used, such as ethylene vinyl acetate, polyanhydrides, polyglycolic acid, collagen, polyorthoesters, and polylacetic acid. Methods for preparation of such formulations will be apparent to those skilled in the art. The materials may also be obtained commercially from Alza Corporation.

The active compound may be administered orally, intravenously or intraperitoneally by infusion or injection. Solutions of the active compound or its salts may be prepared in water, optionally mixed with a non-toxic surfactant. Dispersions may be prepared in glycerol, liquid polyethylene glycols, triacetin, mixtures thereof, and in oils. Under ordinary conditions of storage and use, these preparations contain a preservative to prevent growth of microorganisms.

Pharmaceutical dosage forms suitable for injection or infusion may include sterile aqueous solutions or dispersions or sterile powders comprising the active ingredient which are adapted for the extemporaneous preparation of sterile injectable or infusible solutions or dispersions, optionally encapsulated in liposomes. The ultimate dosage form is optionally sterile, fluid, and stable under conditions of manufacture and storage. The liquid carrier or vehicle may be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity may be maintained, for example, by formation of liposomes, by the maintenance of the required particle size (in the case of dispersions) or by use of one or more surfactants. Microbial growth may be prevented using various antibacterial and antifungal agents, for example, parabens, chlorobutanol, phenol, sorbic acid, thimerosal, and the like. In many cases, it will be preferable to include isotonic agents, for example, sugars, buffers, or sodium chloride. Prolonged absorption of the injectable compositions may be achieved using agents which delay absorption, for example, aluminum monostearate and gelatin.

Liposomal suspensions (including liposomes targeted to infected cells with monoclonal antibodies to viral antigens) are also preferred as pharmaceutically acceptable carriers. These may be prepared according to methods known to those skilled in the art, for example, as described in U.S. Pat. No. 4,522,811. For example, liposome formulations may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compound is then introduced into the container. The container is then swirled by hand to free lipid material from the sides of the container and to disperse lipid aggregates, thereby forming the liposomal suspension.

Sterile injectable solutions are prepared by incorporating the active compound in the required amount in an appropriate solvent, optionally with one or more of the other ingredients enumerated above, followed by filter sterilization. In the case of sterile powders for preparation of sterile injectable solutions, preferred methods of preparation include vacuum drying and the freeze drying techniques, which yield a powder of the active ingredient and any additional desired ingredient present in the previously sterile-filtered solution(s).

A pharmaceutical composition of the invention may be prepared, packaged, or sold in a formulation suitable for rectal administration. Such a composition may be in the form of, for example, a suppository, a retention enema preparation, and a solution for rectal or colonic irrigation.

Suppository formulations may be made by combining the active ingredient with a non-irritating pharmaceutically acceptable excipient which is solid at ordinary room temperature (i.e. about 20° C.) and which is liquid at the rectal temperature of the subject (i.e. about 37° C. in a healthy human). Suitable pharmaceutically acceptable excipients include, but are not limited to, cocoa butter, polyethylene glycols, and various glycerides. Suppository formulations may further comprise various additional ingredients including, but not limited to, antioxidants and preservatives.

Retention enema preparations or solutions for rectal or colonic irrigation may be made by combining the active ingredient with a pharmaceutically acceptable liquid carrier. As is well known in the art, enema preparations may be administered using, and may be packaged within, a delivery device adapted to the rectal anatomy of the subject. Enema preparations may further comprise various additional ingredients including, but not limited to, antioxidants and preservatives.

A pharmaceutical composition of the invention may be prepared, packaged, or sold in a formulation suitable for vaginal administration. Such a composition may be in the form of, for example, a suppository, an impregnated or coated vaginally-insertable material such as a tampon, a douche preparation, or a solution for vaginal irrigation.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e. such as with a physiologically degradable material), and methods of absorbing an aqueous or oily solution or suspension into an absorbent material, with or without subsequent drying.

Douche preparations or solutions for vaginal irrigation may be made by combining the active ingredient with a pharmaceutically acceptable liquid carrier. As is well known in the art, douche preparations may be administered using, and may be packaged within, a delivery device adapted to the vaginal anatomy of the subject. Douche preparations may further comprise various additional ingredients including, but not limited to, antioxidants, antibiotics, antifungal agents, and preservatives.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally, the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration, e.g., have an average diameter in the range from about 0.1 to about 200 nanometers.

The formulations described herein as being useful for pulmonary delivery are also useful for intranasal delivery of a pharmaceutical composition of the invention. Another formulation suitable for intranasal administration is a coarse powder comprising the active ingredient and having an average particle from about 0.2 to 500 micrometers. Such a formulation is administered in the manner in which snuff is taken i.e. by rapid inhalation through the nasal passage from a container of the powder held close to the nose.

Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for ophthalmic administration. Such formulations may, for example, be in the form of eye drops including, for example, a 0.1-1.0% (w/w) solution or suspension of the active ingredient in an aqueous or oily liquid carrier. Such drops may further comprise buffering agents, salts, or one or more other of the additional ingredients described herein. Other ophthalmalmically-administrable formulations which are useful include those which comprise the active ingredient in microcrystalline form or in a liposomal preparation.

For topical administration, the present compounds can be applied in pure form, i.e., as a liquid. However, it will generally be desirable to administer the compounds to the skin as compositions or formulations, in combination with a dermatologically acceptable carrier, which may be a solid or a liquid.

Useful solid carriers include finely divided solids such as talc, clay, microcrystalline cellulose, silica, alumina, and the like. Useful liquid carriers include water, alcohols, glycols, and blends of two or more of these, in which the present compounds can be dissolved or dispersed at effective levels, optionally with the aid of non-toxic surfactants. Adjuvants such as fragrances and additional antimicrobial agents can be added to optimize properties for a given use. The resulting liquid compositions can be applied using absorbent pads, used to impregnate bandages or other dressings, or sprayed onto the affected area using pump-type or aerosol sprayers.

Thickeners such as synthetic polymers, fatty acids, fatty acid salts and esters, fatty alcohols, modified celluloses, or modified mineral materials can also be employed with liquid carriers to form spreadable pastes, gels, ointments, soaps, and the like, for application directly to the skin of the user.

Examples of useful dermatological compositions which can be used to deliver the compounds of the invention to the skin are disclosed in Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Accordingly, the invention includes pharmaceutical compositions comprising one or more compounds described herein or any combination thereof, or a pharmaceutically acceptable salt thereof, in combination with a pharmaceutically acceptable carrier.

In one embodiment, the pharmaceutical composition is adapted for oral, topical, or parenteral administration to a mammal, such as a human, and comprises one or more compounds of the invention, or any combination thereof, or a pharmaceutically acceptable salt thereof.

The drug can be administered as a salt or prodrug that upon administration to the recipient is capable of providing directly or indirectly the parent compound, or that exhibits activity itself. Further, the modifications can affect the biological activity of the compound, in some cases increasing the activity over the parent compound. This can easily be assessed by preparing the salt or prodrug and testing its antiviral activity according to the methods described herein, or other methods known to those skilled in the art.

A preferred mode of administration of the active compound is oral. Oral compositions will generally include an inert diluent or an edible carrier. They may be enclosed in gelatin capsules or compressed into tablets. For the purpose of oral therapeutic administration, the active compound can be incorporated with excipients and used in the form of tablets, troches, or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or other enteric agents.

The compound can be administered as a component of an elixir, suspension, syrup, wafer, chewing gum or the like. A syrup may contain, in addition to the active compounds, sucrose as a sweetening agent and certain preservatives, dyes and colorings and flavors.

The compound or a pharmaceutically acceptable prodrug or salts thereof can also be mixed with other active materials that do not impair the desired action, or with materials that supplement the desired action, such as antibiotics, antifungals, anti-inflammatories, or other antivirals, including other nucleoside compounds. Solutions or suspensions used for parenteral, intradermal, subcutaneous, or topical application can include the following components: a sterile diluent such as water for injection, saline solution, fixed oils, polyethylene glycols, glycerine, propylene glycol or other synthetic solvents; antiacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation can be enclosed in ampoules, disposale syringes or multiple dose vials made of glass or plastic. If administered intravenously, useful carriers are physiological saline or phosphate buffered saline (PS).

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters and polylactic acid. Methods for preparation of such formulations will be apparent to those skilled in the art. The materials can also be obtained commercially from Alza Corporation.

Liposomal suspensions (including liposomes targeted to infected cells with monoclonal antiodies to viral antigens) are also preferred as pharmaceutically acceptable carriers. These may be prepared according to methods known to those skilled in the art, for example, as described in U.S. Pat. No. 4,522,811 (which is incorporated herein by reference in its entirety). For example, liposome formulations may be prepared by dissolving appropriate lipid(s) (such as stearoyl phosphatidyl ethanolamine, stearoyl phosphatidyl choline, arachadoyl phosphatidyl choline, and cholesterol) in an inorganic solvent that is then evaporated, leaving behind a thin film of dried lipid on the surface of the container. An aqueous solution of the active compound or its monophosphate, diphosphate, and/or triphosphate derivatives is then introduced into the container. The container is then swirled by hand to free lipid material from the sides of the container and to disperse lipid aggregates, thereby forming the liposomal suspension.

Useful dosages of the compounds for inclusion in the pharmaceutical compositions of the invention can be determined by comparing in vitro activity and in vivo activity of the compounds in appropriate animal models. Methods for the extrapolation of effective dosages in mice and other animal models to humans are known in the art (see, for example U.S. Pat. No. 4,938,949).

The concentration of the compound(s) in a liquid composition, such as a lotion, will, for example, range from about 0.1% to about 95% by weight, preferably from about 0.5% to about 25% by weight. The concentration in a semi-solid or solid composition such as a gel or a powder will, for example, range from about 0.1% to 100% by weight, preferably about 0.5% to about 5% by weight. Single doses for intravenous injection, subcutaneous, intramuscular or topical administration, infusion, ingestion or suppository will generally be from about 0.001 to about 5000 mg, and be administered from about 1 to about 3 times daily, to yield levels of about 0.01 to about 500 mg/kg, for adults.

The invention also includes one or more compounds disclosed herein, or any combination thereof, or salt thereof, in an amount effective to inhibit togavirus, coronavirus or herpes virus viral replication in a host. The compound can be useful for inhibiting virus replication in a cell or neutralization (i.e. inactivation) of extracellular virus.

As used herein, to inhibit viral replication in a host means to reduce the virus load in a host to a level which is lower than the level of the virus load in an otherwise identical host which was not administered the compound. Preferably, virus load in a mammal is reduced by about 1 to 12 log₁₀ or more relative to an otherwise identical mammal which was not administered the compound. Virus load in a mammal can be assessed by a number of methods known in the art such as, for example, obtaining a tissue or fluid sample from the mammal and assessing the amount of virus or viral components in the mammal contained therein using technology which is either virological, immunological, biochemical or molecular biological in nature and which is well known to the skilled artisan and which are described elsewhere herein. Inhibition of viral replication in a cell is assessed using similar or identical assays as those used to assess virus load in a mammal.

The invention also includes a kit for administering a compound of the invention, a pharmaceutically acceptable salt thereof, or a pharmaceutical composition, to a host for treatment of a togavirus, herpes virus or coronavirus viral infection. Preferably, the host is a human. The kit comprises one or more compounds of the invention, or a combination thereof, and optionally an instructional material, which describes adventitially administering the composition to the mammal by any of the routes of administration described herein. In another embodiment, this kit comprises a (preferably sterile) solvent suitable for dissolving or suspending the composition of the invention prior to administering the compound to the mammal.

As used herein, an “instructional material” includes a publication, a recording, a diagram, or any other medium of expression which can be used to communicate the usefulness of the composition of the invention in the kit for any one or more of the following: effecting treatment of a viral infection in a mammal or in a cell; alleviation or treatment of the symptoms of a viral infection in the mammal. The instructional material of the kit of the invention may, for example, be affixed to a container which contains the composition of the invention or be shipped together with a container which contains the composition. Alternatively, the instructional material may be shipped separately from the container with the intention that the instructional material and the composition be used cooperatively by the recipient.

The invention includes methods for treatment of a togavirus, herpes virus or coronavirus viral infection in a host. The methods comprise administering to the host one or more compounds disclosed herein, or any combination thereof, or a pharmaceutically acceptable salt thereof, in an amount effective to treat the virus infection. The compound may be administered by any of the methods described herein. Preferably, the host is a human. Methods for testing the antiviral activity of a compound in-vitro are known to the skilled artisan, and are described, for example, in Kucera et al., 1990, AIDS Res. and Human Retrovir. 6:494.

The invention further includes methods of using one or more compounds, or any combination thereof, or a pharmaceutically acceptable salt thereof, in medical therapy (preferably for use in treating a virus infection) or for the manufacture of a medicament useful for the treatment of a virus infection.

The invention also includes methods of inhibiting togavirus, herpes virus or coronavirus viral replication in a cell. The methods comprise administering to the cell one or more compounds disclosed herein or any combination thereof, or a pharmaceutically acceptable salt thereof, in an amount effective to inhibit viral replication in the cell. Inhibition of viral replication in a cell, as used herein, is a reduction in virus replication in a cell to a level lower than the level in an otherwise identical cell, which was not administered the compound of the invention. Preferably, the reduction in viral replication is by about 90% to about 99.9% relative to the otherwise identical cell, which was not administered the compound of the invention. The level of viral replication in a cell can be assessed by any one of the methods known to the skilled artisan described herein. For example, the level of viral replication in a cell can be assessed by evaluating the number of viral particles or amount of a viral component, such as a viral protein, a viral enzyme, or viral nucleic acid, in the cell or in fluid or debris associated with the cell. The number of infectious virus particles in a cell can be evaluated, for example, in a plaque assay. The level of a viral component such as a viral protein or enzyme in a cell can be evaluated using standard analytical techniques of protein biochemistry, such as, for example, using an activity assay for a viral enzyme, or using Western blotting or quantitative gel electrophoresis for a viral protein. Viral nucleic acid levels in a cell can be evaluated using standard analytical techniques such as Northern blotting and Southern Blotting or quantitation by polymerase chain reaction (PCR).

The invention includes methods for treatment of a togavirus, coronavirus,or herpes virus infection in a host. The methods comprise administering to the host one or more compounds of having a structure described herein, or any combination thereof, or a pharmaceutically acceptable salt thereof, in an amount effective to treat the virus infection. The compound may be administered by any of the methods described herein. Preferably, the host is a human.

The invention also includes methods of treating a togavirus, coronavirus or herpes virus infection in a host by contacting the virus in vitro, in vivo or ex-vivo with one or more compounds of the invention, or any combination thereof, or a pharmaceutically acceptable salt thereof, in an amount effective to treat the viral infection (e.g. to inhibit virus replication, infectivity, life cycle processes or pathogenesis). Methods for testing the antiviral activity of a compound in-vitro are known to the skilled artisan, and are described, for example, in Kucera et al., 1990, AIDS Res. and Human Retrovir. 6:494.

Drug Administration and Combination and Alternation Therapy

The active compounds can be administered in combination, alternation or sequential steps with another anti-togavirus, anti-herpes virus and/or anti-coronavirus agent. In combination therapy, effective dosages of two or more agents are administered together, whereas in alternation or sequential-step therapy, an effective dosage of each agent is administered serially or sequentially. The dosages given will depend on absorption, inactivation and excretion rates of the drug as well as other factors known to those of skill in the art. It is to be noted that dosage values will also vary with the severity of the condition to be alleviated. It is to be further understood that for any particular subject, specific dosage regimens and schedules should be adjusted over time according to the individual need and the professional judgment of the person administering or supervising the administration of the compositions. In some embodiments, an anti-togavirus, anti-herpes virus and/or anti-coronavirus agent compound that exhibits an EC₅₀ of 10-15 μM or less, or preferably less than 1-5 μM, is desirable.

It is possible that drug-resistant variants of togavirus, herpes virus and/or coronavirus can emerge after prolonged treatment with an antiviral agent. Drug resistance most typically occurs by mutation of a gene that encodes for an enzyme used in viral replication. The efficacy of a drug against the viral infection can be prolonged, augmented, or restored by administering the compound in combination or alternation with a second, and perhaps third, antiviral compound that induces a different mutation from that caused by the principle drug. Alternatively, the pharmacokinetics, biodistribution or other parameter of the drug can be altered by such combination or alternation therapy. In general, combination therapy is typically preferred over alternation therapy because it induces multiple simultaneous stresses on the virus.

Any of the viral treatments described in the Background of the Invention can be used in combination or alternation with the compounds described in this specification.

Nonlimiting examples include:

(1) an interferon and/or ribavirin (Battaglia, A. M. et al., Ann. Pharmacother. 34:487-494, 2000); Berenguer, M. et al. Antivir. Ther. 3(Suppl. 3):125-136, 1998);
(2) substrate-based NS3 protease inhibitors (Attwood et al., Antiviralpeptide derivatives, PCT WO 98/22496, 1998; Attwood et al., Antiviral Chemistry and Chemotherapy 10.259-273, 1999; Attwood et al., Preparation and use of amino acid derivatives as anti-viral agents, German Patent Publication DE 19914474; Tung et al. Inhibitors of serine proteases, PCT WO 98/17679), including alphaketoamides and hydrazinoureas, and inhibitors that terminate in an electrophile such as a boronic acid or phosphonate. (Llinas-Brunet et al, PCT WO 99/07734).
(3) non-substrate-based inhibitors such as 2,4,6-trihydroxy-3-nitro-benzamide derivatives (Sudo K. et al., Biochemical and Biophysical Research Communications, 238:643-647, 1997; Sudo K. et al. Antiviral Chemistry and Chemotherapy 9:186, 1998), including RD3-4082 and RD3-4078, the former substituted on the amide with a 14 carbon chain and the latter processing a para-phenoxyphenyl group;
(4) thiazolidine derivatives which show relevant inhibition in a reverse-phase HPLC assay with an NS3/4A fusion protein and NS5A/5 substrate (Sudo K. et al., Antiviral Research 32:9-18, 1996), especially compound RD-1-6250, possessing a fused cinnamoyl moiety substituted with a long alkyl chain, RD4 6205 and RD4 6193;
(5) thiazolidines and benzanilides identified in Kakiuchi N. et al., J. FEBS Letters 421:217-220; Takeshita N. et al., Analytical Biochemistry 247:242-246, 1997;
(6) a phenanthrenequinone possessing activity against viral protease in a SDS-PAGE and autoradiography assay isolated from the fermentation culture broth of Streptomyces sp., Sch 68631 (Chu M. et al., Tetrahedron Letters 37:7229-7232, 1996), and Sch 351633, isolated from the fungus Penicillium griseofulvum, which demonstrates activity in a scintillation proximity assay (Chu M. et al., Bioorganic and Medicinal Chemistry Letters 9:1949-1952);
(7) selective NS3 inhibitors based on the macromolecule elgin c, isolated from leech (Qasim M. A. et al., iochemistry 36:1598-1607, 1997);
(8) antisense phosphorothioate oligodeoxynucleotides (S-ODN) complementary to sequence stretches in the 5′ non-coding region (NCR) of the virus (Alt M. et al., Hepatology 22:707-717, 1995), or nucleotides 326-348 comprising the 3′ end of the NCR and nucleotides 371-388 located in the core coding region of the IICV RNA (Alt M. et al., Archives of Virology 142:589-599, 1997; Galderisi U. et al., Journal of Cellular Physiology 181:251-257, 1999);
(9) inhibitors of IRES-dependent translation (Ikeda N et al., Japanese Patent Publication JP-08268890; Kai Y. et al. Prevention and treatment of viral diseases, Japanese Patent Publication JP-10101591);
(10) nuclease-resistant ribozymes. (Maccjak D. J. et al., Hepatology 30 Abstract 995, 1999); and
(11) other miscellaneous compounds including 1-amino-alkylcyclohexanes (U.S. Pat. No. 6,034,134 to Gold et al.), alkyl lipids (U.S. Pat. No. 5,922,757 to Chojkier et al.), vitamin E and other antioxidants (U.S. Pat. No. 5,922,757 to Chojkier et al.), squalene, amantadine, bile acids (U.S. Pat. No. 5,846,964 to Ozeki et al.), N-(phosphonoacetyl)-L-aspartic acid, (U.S. Pat. No. 5,830,905 to Diana et al.), benzenedicarboxamides (U.S. Pat. No. 5,633,388 to Diana et al.), polyadenylic acid derivatives (U.S. Pat. No. 5,496,546 to Wang et al.), 2′,3′-dideoxyinosine (U.S. Pat. No. 5,026,687 to Yarchoan et al.), and benzimidazoles (U.S. Pat. No. 5,891,874 to Colacino et al.); and
(12) PEGASYS (pegylated interferon alfa-2a) by Roche, INFERGEN (interferon alfacon-1) by InterMune, OMNIFERON (natural interferon) by Viragen, ALUFERON by Human Genome Sciences, REIF (interferon beta-1a) by Ares-Serono, Omega Interferon by BioMedicine, Oral Interferon Alpha by Amarillo Biosciences, Interferon gamma-b1 by InterMune, Interleukin-10 by Schering-Plough, IP-501 by Interneuron, Merimeodi VX-497 by Vertex, AMANTADINE (Symmetrel) by Endo Las Solvay, HEPTAZYME by RPI, IDN-6556 by Idun Pharma., XTL-002 by XTL., CIVACIR by NAI, LEVOVIRIN by ICN, VIRAMIDINE by ICN, ZADAXIN (thymosin alfa-1) by Sci Clone, CEPLENE (histamine dihydrochloride) by Maxim, VX 950/LY 570310 by Vertex/Eli Lilly, ISIS 14803 by Isis Pharmaceutical/Elan, IDN-6556 by Idun Pharmaceuticals, Inc. and JTK 003 by AKROS Pharma.

The present invention is described by way of illustration in the following examples. It will be understood by one of ordinary skill in the art that these examples are in no way limiting and that variations of detail can be made without departing from the spirit and scope of the present invention.

### EXAMPLES

### Example 1

### Anti-SARS CoV Activity

The ability of active compounds to inhibit the SARS CoV was determined using a neutral red assay. Vero cells were infected with the Urbani strain of SARS CoV. Serial concentrations of test compound were incubated in the presence of the infected cells. Cell survival was quantitated by staining of live cells with a neutral red solution. Toxicity was determined by incubating uninfected Vero cells in the presence of serial concentrations of the test compound.

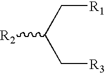

| | | | 50% Endpoint (μM) | | |
|---|---|---|---|---|---|
| | | | Effective Conc. | Inhibitory | |
| Cmpd: | | | (EC₅₀) Against | Cone. (IC₅₀) | |
| R₁ | R₂ | R₃ | SARS CoV | Cell Growth | SI^{a} |
| NHCOC₁₁H₂₃ | OC₃H₆CH₃ | PC^{b} | 3 μg/mL | 40 μg/mL | 13 |

^{a}SI = Selectivity Index (IC₅₀ Cell Growth divided by EC₅₀).
^{b}PC = Phosphocholine [OPO₃⁻CH₂CH₂N⁺(CH₃)₃]

### Example 2

### Anti-Varicella Zoster Virus Activity

The ability of active compounds to inhibit the varicella zoster virus was determined using a CPE (virus-induced cytopathic effects) inhibition assay. Human foreskin fibroblast cells were infected with varicella zoster virus. Serial concentrations of test compound were incubated in the presence of the infected cells. The CPE reduction of the virus-infected wells and the percentage cell viability of uninfected drug control wells were determined. The EC₅₀ and the TC₅₀ were calculated.

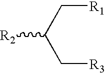

| | 50% Endpoint (μM) | |
|---|---|---|
| | Effective | |
| | Conc. | |
| Compound: | (EC₅₀)^{c} | Inhibitory |
| | Against | Cone. (IC₅₀) |

| | | | | | |
|---|---|---|---|---|---|
| R₁ | R₂ | R₃ | VZV | Cell Growth | SI^{a} |

| | | | | | |
|---|---|---|---|---|---|
| NHCOC₉H₁₉ | OC₉H₁₈CH₃ | PC^{b} | 0.48 μg/mL | 75 μg/mL | 156 |

^{a}SI = Selectivity Index (IC₅₀ Cell Growth divided by EC₅₀).
^{b}PC = Phosphocholine [OPO₃⁻CH₂CH₂N⁺(CH₃)₃]

### Example 3

### Preparation of Pharmaceutical Compositions

Alkylamidophosphocholine compounds are synthesized as described in Ouyang et al., J. Med. Chem., 45:2857-2866 (2002), U.S. Pat. No. 5,614,548, U.S. Pat. No. 5,962,437, or U.S. Pat. No. 5,770,584.

In particular, the 3-alkylamido-2-alkoxypropylphosphocholine is obtained by reacting commercially available 3-amino-1,2,-propanediol with the appropriate acid halide, such as an acid chloride, and/or anhydride. The primary alcohol is protected, and the secondary alcohol is alkylated, for example with an alkyl halide, such as an alkyl bromide. The primary alcohol is deprotected and reacted with a 2-haloalkyl dihalophosphate, such as 2-bromoethyl dichlorophosphate, and a base, such as trimethylamine, to obtain the 3-alkylamido-2-alkoxypropylphosphocholine compound.

3-dodecylamido-2-ethoxypropylphosphocholine is synthesized as shown in FIG. 2, and as described in Ouyang et al. FIG. 2 describes the chemical synthesis of R, S, and racemic 3-dodecylamido-2-ethoxypropylphosphocholine and shows there is a chiral center on the C-2 position of the three carbon backbone. 3-nonylamido-2-ethoxypropyl-phosphocholine also is synthesized according to this method.

1. A method for treating a host infected with a togavirus, a coronavirus or a herpes virus, comprising administering an anti-viral effective amount of a compound, or a pharmaceutically acceptable salt or prodrug thereof, having a structure of Formula I: 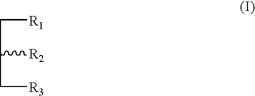
wherein:
R₁ is —NHC(O)Y, where Y is C₁-C₂₂ alkyl, C₂-C₂₂ alkenyl, or C₂-C₂₂ alkynyl;
R₂ is —OX, where X is C₁-C₂₂ alkyl, C₂-C₂₂ alkenyl, C₂-C₂₂ alkynyl; and
R₃ is phosphocholine;
optionally with a pharmaceutically acceptable carrier or diluent.

2. The method of claim 1, wherein
Y is C₁-C₁₄ alkyl, C₂-C₁₄ alkenyl, or C₂-C₁₄ alkynyl; and
X is C₁-C₁₄ alkyl, C₂-C₁₄ alkenyl, or C₂-C₁₄ alkynyl.

3. The method of claim 1 wherein:
Y is —C₁₁H₂₃, —C₁₀H₂₁ or —C₉H₁₉; and
X is —CH₂CH₃, —(CH₂)₂CH₃, —(CH₂)₃CH₃, or —CH₁₀CH₂₁.

4. The method of claim 1, wherein Y is —C₁₁H₂₃ and X is C₁-C₅ alkyl.

5. The method of claim 1, wherein Y is —C₉H₁₉ and X is C₉-C₁₁ alkyl.

6. The method of claim 1, wherein the compound is: 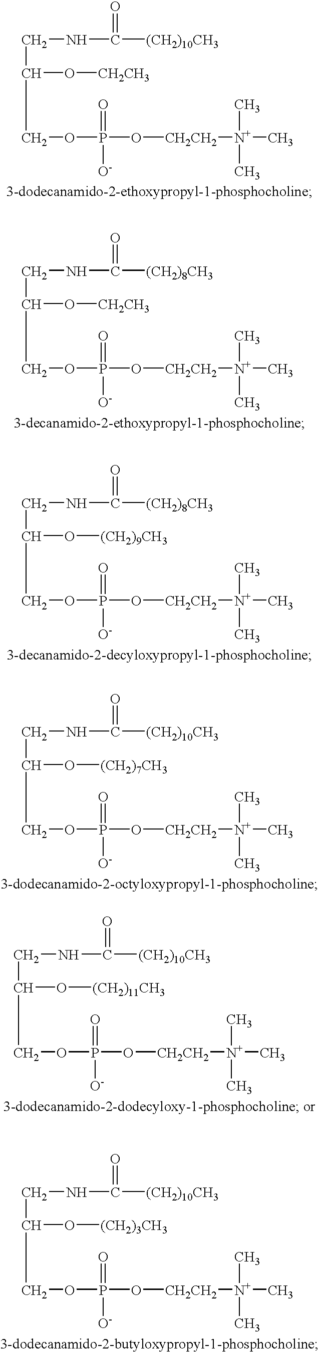
or a combination thereof.

7. The method of claim 1, wherein the virus is a coronavirus.

8. The method of claim 7, wherein the coronavirus is SARS-CoV.

9. The method of claim 1, wherein the virus is a herpes virus.

10. The method of claim 9, wherein the herpes virus is varicella zoster virus.

11. The method of claim 9, wherein the herpes virus is cytomegalovirus.

12. The method of claim 1, wherein the host is a mammal.

13. The method of claim 1, wherein the host is a human.

14. A method for treating a host infected with a togavirus, herpes virus or coronavirus, comprising administering an anti-viral effective amount of a compound, or a pharmaceutically acceptable salt or prodrug thereof, having a structure of Formula II: 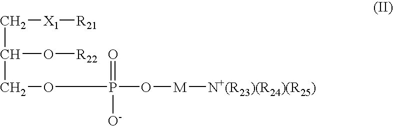
wherein:
M is C₂-C₄ alkyl;
X₁ is —S—, —O—, —NH—, or —NHC(O)—;
R₂₁ is —C₁-C₂₀ straight chain alkyl, —C₂-C₂₀ straight chain alkylene containing not more than four double bonds, or aryl;
R₂₂ is —C₁-C₂₀ straight chain alkyl, —C₂-C₂₀ straight chain alkylene containing not more than four double bonds, or aryl; and
R₂₃, R₂₄, and R₂₅ are each independently either hydrogen, methyl, ethyl, propyl, or isopropyl;
optionally with a pharmaceutically acceptable carrier or diluent.

15. The method of claim 14 wherein:
M is —CH₂CH₂—;
X₁ is —S—, —O—, —NH—, or —NHC(O)—;
R₂₁ is C₁-C₁₆ straight chain alkyl, or —C₂-C₁₆ straight chain alkylene containing not more than one double bond;
R₂₂ is C₁-C₁₆ straight chain alkyl, or —C₂-C₁₆ straight chain alkylene containing not more than one double bond; and
R₂₃, R₂₄, and R₂₅ are each independently hydrogen or methyl.

16. The method of claim 14 wherein:
R₂₂ is C₁-C₅ straight chain alkyl, or —C₂-C₅ straight chain alkylene containing not more than one double bond.

17. The method of claim 15, wherein R₂₁ is —C₉-C₁₂alkyl, and R₂₂ is —C₁-C₁₂ alkyl.

18. The method of claim 15, wherein R₂₁ is —C₉-C₁₂ alkyl, and R₂₂ is —C₁-C₅ alkyl.

19. The method of claim 15, wherein R₂₁ is —C₉-C₁₂ alkyl, and R₂₂ is —C₈-C₁₂ alkyl.

20. The method of claim 14, wherein the virus is a coronavirus.

21. The method of claim 20, wherein the coronavirus is SARS-CoV.

22. The method of claim 14, wherein the virus is a herpes virus.

23. The method of claim 22, wherein the herpes virus is varicella zoster virus.

24. The method of claim 22, wherein the herpes virus is cytomegalovirus.

25. The method of claim 14, wherein the host is a mammal.

26. The method of claim 14, wherein the host is a human.

27. A method for treating a host infected with a togavirus, herpes virus or coronavirus comprising administering an anti-viral effective amount of a compound, or a pharmaceutically acceptable salt or prodrug thereof, having a structure of Formula III: 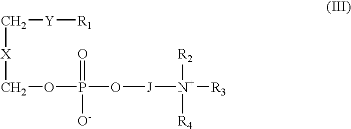
wherein:
Y is —S—, —O—, —NH—, —N(CH₃)—, —NHC(O)—, or —N(CH₃)C(O)—;
R₁ is C₁-C₁₈ alkyl, C₂-C₁₈ alkenyl, C₂-C₁₈ alkynyl or aryl;
X is a covalent bond or methylene that is optionally substituted with hydroxyl, C₁-C₂₀ alkyl, —O—(C₁-C₂₀ alkyl), —S—(C₁-C₂₀ alkyl), —(C(O)N(C₁-C₂₀ alkyl), C₂-C₂₀ alkenyl, —O—(C₂-C₂₀ alkenyl), —S—(C₂-C₂₀ alkenyl), —(C(O)N(C₂-C₂₀ alkenyl), C₂-C₂₀ alkynyl, —O—(C₂-C₂₀ alkynyl), —S—(C₂-C₂₀ alkynyl) or —(C(O)N(C₂-C₂₀ alkynyl);
J is C₁-C₄ alkyl optionally substituted one to three times with methyl or ethyl; and
R₂, R₃, and R⁴ are H or C₁-C₃ alkyl;
optionally with a pharmaceutically acceptable carrier or diluent.

28. The method of claim 27 wherein:
Y is —NHC(O)—;
R₁ is —C₆-C₁₈ alkyl;
X is —CH—O—(C₁-C₁₈ alkyl) or —CH—O—(C₁-C₁₈ alkenyl);
J is —CH₂CH₂—; and
R₂, R₃, and R₄ are each methyl.

29. The method of claim 28, wherein X is —CH—O—(C₁-C₅ alkyl) or —CH—O—(C₂-C₅ alkenyl).

30. The method of claim 28, wherein R₁ is —C₈-C₁₂ alkyl and X is —CH—O—(C₁-C₅ alkyl) or —CH—O—(C₂-C₅ alkenyl).

31. The method of claim 28, wherein R₁ is —C₈-C₁₂ alkyl and X is —CH—O—(C₈-C₁₂ alkyl) or —CH—O—(C₈-C₁₂ alkenyl).

32. The method of claim 27, wherein the virus is a coronavirus.

33. The method of claim 32, wherein the coronavirus is SARS-CoV.

34. The method of claim 27, wherein the virus is a herpes virus.

35. The method of claim 34, wherein the herpes virus is varicella zoster virus.

36. The method of claim 34, wherein the herpes virus is cytomegalovirus.

37. The method of claim 27, wherein the host is a mammal.

38. The method of claim 27, wherein the host is a human.

39. A method for treating a host infected with a coronavirus,herpes virus or togavirus, comprising administering an anti-viral effective amount of a compound, or a pharmaceutically acceptable salt or prodrug thereof, having a structure of Formula IV: 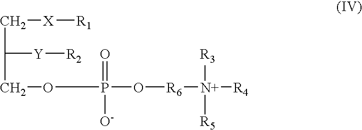
wherein:
R₁ is a C₆-C₁₈ alkyl, C₆-C₁₈ alkenyl, or C₆-C₁₈ alkynyl that is optionally substituted from 1 to 5 times with —OH, —COOH, oxo, amino, or aryl;
X is —NHC(O)—, —N(CH₃)C(O)—, —C(O)NH—, —C(O)N(CH₃)—, —S—, —S(O)—, —(SO₂)—, —O—, —NH—, and —N(CH₃)—;
R₂ is a C₁-C₁₄ alkyl, C₂-C₁₄ alkenyl, or C₂-C₁₄ alkynyl that is optionally substituted from 1 to 5 times with —OH, —COOH, oxo, amino, or aryl;
Y is —NHC(O)—, —N(CH₃)C(O)—, —C(O)NH—, —C(O)N(CH₃)—, —S—, —S(O)—, —(SO₂)—, —O—, —NH—, —N(CH₃)—, or —OC(O)—;
R₆ is a C₂-C₆ alkyl; C₂-C₆ alkenyl, or C₂-C₆ alkynyl; and
R₃, R₄, and R₅ are independently methyl or ethyl, or R₃ and R⁴ together form an aliphatic or heterocyclic ring having five or six ring atoms and R₅ is methyl or ethyl;
optionally with a pharmaceutically acceptable carrier or diluent.

40. The method of claim 39 wherein
R₂ is C₁-C₁₄ alkyl, C₂-C₁₄ alkenyl, or C₂-C₁₄ alkenyl;
R⁶ is CH₂CH₂; and
R₃, R₄, and R₅ are each independently CH₃.

41. The method of claim 40, wherein R₂ is —C₁-C₅ alkyl or —C₁-C₅ alkenyl.

42. The method of claim 40, wherein R₁ is —C₈-C₁₂ alkyl and R₂ is —C₈-C₁₂ alkyl.

43. The method of claim 40, wherein R₁ is —C₈-C₁₂ alkyl and R₂ is —C₁-C₅ alkyl.

44. The method of claim 40, wherein R¹ is —C₈-C₁₂ alkyl and R₂ is —C₈-C₁₂ alkyl.

45. The method of claim 39, wherein:
X is —NHC(O)—, —N(CH₃)C(O)—, —C(O)NH—, or —C(O)N(CH₃)—; and
Y is —O—, —NH—, or —N(CH₃)—.

46. The method of claim 39, wherein the virus is a coronavirus.

47. The method of claim 46, wherein the coronavirus is SARS-CoV.

48. The method of claim 39, wherein the virus is a herpes virus.

49. The method of claim 48, wherein the herpes virus is varicella zoster virus.

50. The method of claim 47, wherein the herpes virus is cytomegalovirus.

51. The method of claim 39, wherein the host is a mammal.

52. The method of claim 39, wherein the host is a human.

53. A method for treating a host infected with a coronavirus,herpes virus or togavirus, comprising administering an anti-viral effective amount of a compound, or a pharmaceutically acceptable salt or prodrug thereof, having a structure of Formula AA-1: 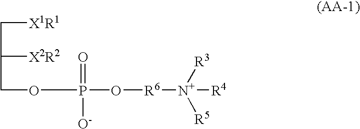
wherein:
X¹ is —NHC(O)—;
X² is —O—;
R¹ is —C₁-C₂₂ alkyl;
R² is —C₁-C₂₂ alkyl;
R⁶ is —CH₂CH₂; and
R³, R⁴ and R⁵ are methyl.

54. The method of claim 53, wherein:
R¹ is —CH₃, —CH₂CH₃, —CH₂CH₂CH₃, —CH₂CH₂CH₂CH₃, —CH₂CH₂CH₂CH₂CH₃, —(CH₂)₅CH₃, —(CH₂)₆CH₃, —(CH₂)₇CH₃, —(CH₂)₈CH₃, —(CH₂)₉CH₃, —(CH₂)₁₀CH₃, —(CH₂)₁₁CH₃, —(CH₂)₁₂CH₃ or —(CH₂)₁₃CH₃; and
R² is —CH₃, —CH₂₂CH₃, —CH₂CH₂CH₂CH₃, —CH₂CH₂CH₂CH₂CH₃, —(CH₂)₅CH₃, —(CH₂)₆CH₃, —(CH₂)₇CH₃, —(CH₂)₈CH₃, —(CH₂)₉CH₃, —(CH₂)₁₀CH₃, —(CH₂)₁₁CH₃, —(CH₂)₁₂CH₃ or —(CH₂)₁₃CH₃.

55. The method of claim 53, wherein the host is infected with a coronavirus.

56. The method of claim 55, wherein the coronavirus is SARS-CoV.

57. The method of claim 56, wherein:
R¹ is —(CH₂)₉CH₃, —(CH₂)₁₀CH₃, or —(CH₂)₁₁CH₃; and
R² is —CH₂CH₂CH₃, —CH₂CH₂CH₂CH₃, or —CH₂(CH₂)₃CH₃.

58. The method of claim 56, wherein the compound is:

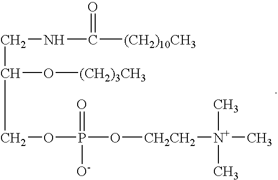

59. The method of claim 53, wherein the host is infected with a herpes virus.

60. The method of claim 59, wherein the herpes virus is varicella zoster virus.

61. The method of claim 60, wherein:
R¹ is —(CH₂)₇CH₃, —(CH₂)₈CH₃, or —(CH₂)₉CH₃;
R² is —(CH₂)₉CH₃, —(CH₂)I₀CH₃, or —(CH₂)₁₁CH₃;

62. The method of claim 60, wherein the compound is:

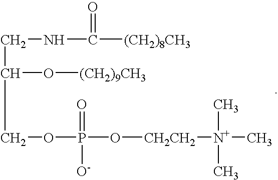

63. The method of claim 59, wherein the herpes virus is cytomegalovirus.

64. The method of claim 1, wherein the virus is a togavirus.

65. The method of claim 1, wherein the compound is administered orally, by inhalation, intravenously, parenterally, intradermally, subcutaneously or topically.
